(19) United States
(12) Patent Application Publication
(10) Pub. No.: US 2025/0059215 A1
(43) Pub. Date: Feb. 20, 2025

(54) Trifluoroborate (BF3) Compositions For Use In Boron Neutron Capture Therapy and Methods Thereof

(71) Applicant: TAE Life Sciences LLC, Irvine, CA (US)

(72) Inventors: Jason C. Quintana, Santa Monica, CA (US); Tioga Martin, Los Angeles, CA (US)

(21) Appl. No.: 18/796,638

(22) Filed: Aug. 7, 2024

Related U.S. Application Data
(60) Provisional application No. 63/628,638, filed on Aug. 7, 2023.

(51) Int. Cl. C07F 5/02; A61K 41/00; A61P 35/00

(52) U.S. Cl. CPC C07F 5/025; A61K 41/0095; A61P 35/00

(57) ABSTRACT

Trifluoroborate (BF₃) Compounds (“BF₃ compounds”) and methods of making BF₃ compounds are disclosed herein. Consequently, the BF₃ compounds can be administered to patients as a Neutron Capture Agent or as a single agent or in combination with other BF₃ compounds and provide a method of treating cancer, immunological disorders, and other disease by utilizing various treatment modalities.

### CROSS-REFERENCE TO RELATED APPLICATIONS

This application claims priority to U.S. Provisional Patent Application No. 63/628,638 filed 7 Aug. 2023, the contents of which are fully incorporated by reference herein.

### STATEMENT OF RIGHTS TO INVENTIONS MADE UNDER FEDERALLY SPONSORED RESEARCH

Not applicable.

### FIELD OF THE INVENTION

The invention described herein relates to the field of boron neutron capture therapy (BNCT).

Specifically, the invention relates to trifluoroborate (BF₃) amino acid compositions which can be used as a vehicle for neutron capture therapy in humans. The invention further relates to the treatment of cancers and other immunological disorders and diseases.

### BACKGROUND OF THE INVENTION

Cancer is the second leading cause of death next to coronary disease worldwide. Millions of people die from cancer every year and in the United States alone cancer kills well over a half-million people annually, with 1.8M new cancer cases diagnosed in 2020 (American Cancer Society). While deaths from heart disease have been declining significantly, those resulting from cancer generally are on the rise. In the early part of the next century, cancer is predicted to become the leading cause of death unless medical developments change the current trend.

Several cancers stand out as having high rates of mortality. In particular, carcinomas of the lung (18.4% of all cancer deaths), breast (6.6% of all cancer deaths), colorectal (9.2% of all cancer deaths), liver (8.2% of all cancer deaths), and stomach (8.2% of all cancer deaths) represent major causes of cancer death for both sexes in all ages worldwide (GLOBOCAN 2018). These and virtually all other carcinomas share a common lethal feature in that they metastasis to sites distant from the primary tumor and with very few exceptions, metastatic disease fatal. Moreover, even for those cancer patients who initially survive their primary cancers, common experience has shown that their lives are dramatically altered. Many cancer patients experience strong anxieties driven by the awareness of the potential for recurrence or treatment failure. Many cancer patients also experience physical debilitations following treatment. Furthermore, many cancer patients experience a recurrence of their disease.

Although cancer therapy has improved over the past decades and survival rates have increased, the heterogeneity of cancer still demands new therapeutic strategies utilizing a plurality of treatment modalities. This is especially true in treating solid tumors at anatomical crucial sites (e.g., glioblastoma, squamous carcinoma of the head and neck and lung adenocarcinoma) which are sometimes limited to standard radiotherapy and/or chemotherapy. Nonetheless, detrimental effects of these therapies are chemo- and radio resistance, which promote loco-regional recurrences, distant metastases and second primary tumors, in addition to severe side-effects that reduce the patients' quality of life.

Neutron Capture Therapy (NCT) is a promising form of radiation therapy. It is a technique that selectively kills tumor cells using boron compound while sparing the normal cells. BNCT relies on the propensity of non-radioactive ¹⁰B isotope to absorb epithermal neutrons that fall into the low energy range of 0.5 keV<Eₙ<30 keV. Following neutron capture, the boron atom undergoes a nuclear fission reaction giving rise to an alpha-particle and a recoiled lithium nucleus (⁷Li) as follows:

$${}^{10} B + n \to {}^{7} Li + {}^{4} He$$

The alpha particle deposits high energy i.e., 150 keV/μm along their short path essentially restricted to a single cell diameter that results in a double strand DNA break followed by cancer cell death by apoptosis. Thus, BNCT integrates a concept of both chemotherapy, targeted therapy, and the gross anatomical localization of traditional radiotherapy.

Even though the conceptual techniques of NCT and specifically Boron Neutron Capture Therapy (BNCT) are well known, the technological limitations associated with this type of treatment have slowed progress. During the early investigations using the research reactors of MIT in 1960's, several dozens of patients were treated using disodium decahydrodecaborate, which was considered less toxic than simple boron compounds used previously yet capable of delivering more boron to the cell. Unfortunately, BNCT studies were halted in the USA due to the severe brain necrosis in the patients undergoing BNCT and the potential harm of using nuclear reactors.

Hiroshi Hatanaka in 1968 re-investigated clinical application of BNCT in Japan using sodium borocaptate (BSH) by directing the beam to surgically exposed intracranial tumor and reported of achieving 58% of 5-year survival rate. In 1987 clinicians in Japan applied BNCT for the treatment of malignant melanoma using boronophenylalanine (BPA) as boron compound. Thus, slow resurgence of BNCT took place albeit limited to the countries with an access to research reactor facilities capable of delivering epithermal neutron beams. Currently, given the technological improvements in both (i) the infusion and delivery of a capture compound, which preferably concentrates in the tumor, and (ii) more abundant and easier access to neutron beam using cyclotrons, there has been a resurgence in NCT treatment methods.

The proton boron fusion reaction relies on the naturally abundant ¹¹B isotope rather than ¹⁰B required for BNCT. Unlike BNCT, three alpha particles are emitted after the fusion reaction between a proton (¹H) and a boron (¹¹B) nucleus: p+¹¹B→3α. The proton beam has the advantage of a Bragg-peak characteristic reducing the normal tissue damage and when combined with proton capture, may improve the efficacy of the proton therapy alone.

Carriers of boron have evolved since the 1950s and are reviewed in NEDUNCHEZHIAN, et. al., J. Clin. & Diag. Res., vol. 10(12) pp. ZE01-ZE04 (December 2016). Briefly, the 1^{st} generation of boron compounds represented by boric acid and its derivatives were either toxic or suffered from low tumor accumulation/retention. BPA and BSH are both considered the 2^{nd} generation compounds that emerged in 1960s. These had significantly lower toxicity and better PK and biodistribution. BPA-fructose complex is considered the 3^{rd} generation compound that is used to treat patients with H&N, glioblastoma and melanoma using BNCT since 1994. BPA-fructose and BSH are the only compounds that are being used in clinic as boron carriers to date although both low and high molecular weight biomolecules such as nucleosides, porphyrins, liposomes, nanoparticles and mAbs have been evaluated for the tumor targeting in preclinical models. The main deficiency of BPA-fructose is relatively low solubility combined with its rapid clearance that prevents achieving high Cₘₐₓ in blood, one of the drivers influencing the tumor uptake.

Additionally, organometallic reagents have become a fundamental necessity for modern day chemists. Since the discovery of the Suzuki-Miyaura reaction, See, KOTHA, et. al., Tetrahedron, 58, 9633 (2002) & MIYAURA, et. al., Chem. Rev. 95, 2457 1995), organoboranes have taken precedent over other organometallic reagents due to their low toxicity and facile carbon-carbon bond formation in the presence of a palladium catalyst. The increased versatility of these motifs relies heavily off of their availability, which are most commonly introduced via trans-metalation or hydroboration. See, for example, PELTER, et. al., Borane Reagents (1988). However, due to the vacant orbital of boron, many alkyl- and alkynyl boranes are not stable under atmospheric conditions. It was not until the 1960's, when potassium organo-trifluoroborates were further explored, did these susceptibilities mitigate.

The first report of an organo-trifluoroborate complex being fabricated was described in 1940, by Fowler and Krauss. See, FOWLER, et. al., J. Am. Chem. Soc. 82:1888 (1960). They reacted a triphenylborane-ammonia complex with tetraalkylammonium fluoride to yield the corresponding tetramethyl- and tetrabutylammonium triphenyl-fluoroborates. Approximately, twenty (20) years later, investigations of converting organostannanes, organosilanes, and dihalogenoboranes into potassium trifluoroborates have shown that the corresponding salts are both nonhygroscopic and highly stable up to 300° C. See, for example, PAWWELKE, et. al., Organomet. Chem. 178:1 (1979). Similar to the prior work in the field, these methods proved to be unsatisfactory due to the toxicity of organotin reagents and the highly reactive and unstable organo-dihalogenoboranes.

Then, in 1967, Thierig and Umland described a nontoxic method to convert hydroxydiphenylborane-ethanolamine complex to the corresponding diphenyldifluoroborate via potassium bifluoride in quantitative yields. See, THIERIG, et. al., Naturwissenschaften 54:563 (1967).

Not until 1995, Vedejs, et al elaborated on utilizing potassium bifluoride in aqueous methanol to achieve high conversion rates of the trivalent boron reagents to the corresponding potassium trifluoroborates. See, for example, VEDEJS, et. al., J. Org. Chem. 60:3020 (1995). Notably, the elucidation of converting trivalent organoboron species to their trifluoroborates provided a nontoxic pathway towards a highly stable boron salt that ultimately affords many commercially available trifluoroborates used today.

From the aforementioned, it will be readily apparent to those skilled in the art that a new treatment paradigm is needed in the treatment of cancers and immunological diseases. By using modern chemical synthesis and modifying natural amino acids with boron, a new disease treatment can be achieved with the overall goal of more effective treatment, reduced side effects, and lower production costs.

Given the current deficiencies associated with NCT, it is an object of the present invention to provide new and improved methods of treating cancer(s), immunological disorders, and other diseases utilizing novel BF₃ compositions as a capture agent in NCT and BNCT treatments.

### SUMMARY OF THE INVENTION

The invention provides for trifluoroborate (BF₃) compositions which have been borylated via chemical synthesis for use as a delivery modality to treat human diseases such as cancer, immunological disorders, including but not limited to rheumatoid arthritis, ankylosing spondylitis, and other cellular diseases, including but not limited to Alzheimer's disease. In certain embodiments, the trifluoroborate (BF₃) compositions are comprised of naturally occurring amino acids such as phenylalanine, leucine, methionine, proline, and any other naturally occurring amino acid set forth in Table I.

In a further embodiment, the invention comprises methods of concentrating Boron in a cell comprising (i) synthesizing a trifluoroborate (BF₃) composition (“BF₃ Comp”); (ii) administering the BF₃ Comp to a patient, and (iii) irradiating the cell with neutrons.

In another embodiment, the present disclosure teaches methods of synthesizing FBH.

In another embodiment, the present disclosure teaches methods of synthesizing FBF.

In another embodiment, the present disclosure teaches methods of synthesizing a class of compounds utilizing L-Phenylalanine mimetics.

In another embodiment, the present disclosure teaches methods of synthesizing a class of compounds utilizing L-Phenylalanine mimetics utilizing Ellman's sulfinamide.

In another embodiment, the present disclosure teaches methods of synthesizing LBH.

In another embodiment, the present disclosure teaches methods of synthesizing LBF.

In another embodiment, the present disclosure teaches methods of synthesizing a class of compounds utilizing L-Leucine mimetics.

In another embodiment, the present disclosure teaches methods of synthesizing a class of compounds utilizing L-Leucine mimetics utilizing Ellman's sulfinamide.

In another embodiment, the present disclosure teaches methods of synthesizing MBH.

In another embodiment, the present disclosure teaches methods of synthesizing MBF.

In another embodiment, the present disclosure teaches methods of synthesizing a class of compounds utilizing L-Methionine mimetics.

In another embodiment, the present disclosure teaches methods of synthesizing a class of compounds utilizing L-Methionine mimetics utilizing Ellman's sulfinamide.

In another embodiment, the present disclosure teaches methods of synthesizing PBH.

In another embodiment, the present disclosure teaches methods of synthesizing PBF.

In another embodiment, the present disclosure teaches methods of synthesizing a class of compounds utilizing L-Proline mimetics.

In another embodiment, the present disclosure teaches methods of synthesizing a class of compounds utilizing L-Proline mimetics utilizing Ellman's sulfinamide.

In another embodiment, the present disclosure teaches methods of Citronella derivatives.

In another embodiment, the present disclosure teaches methods of ((1R)-1-amino-3,7-dimethyloct-6-en-1-yl)boronic acid.

In another embodiment, the present disclosure teaches methods of Potassium ((1R)-1-amino-3,7-dimethyloct-6-en-1-yl)trifluoroborate.

In another embodiment, the present disclosure teaches methods of synthesizing Bortezomib.

In another embodiment, the present disclosure teaches methods of synthesizing Delanzomib.

In another embodiment, the present disclosure teaches methods of synthesizing Ixazomib.

In another embodiment, the present disclosure teaches methods of synthesizing Bortezomib further comprising using LBH as a synthon.

In another embodiment, the present disclosure teaches a synthetic schema for the installation of alpha boronic acids and subsequent trifluoroborates having a chemical structure set forth in FIG. 1.

In another embodiment, the present disclosure teaches methods of synthesizing FBH and FBF having a chemical structure set forth in FIG. 2.

In another embodiment, the present disclosure teaches methods of synthesizing LBH and LBF having a chemical structure set forth in FIG. 7.

In another embodiment, the present disclosure teaches methods of synthesizing MBH and MBF having a chemical structure set forth in FIG. 12.

In another embodiment, the present disclosure teaches methods of synthesizing PBH and PBF having a chemical structure set forth in FIG. 15.

In another embodiment, the present disclosure teaches methods of synthesizing ((1R)-1-amino-3,7-dimethyloct-6-en-1-yl)boronic acid and Potassium ((1R)-1-amino-3,7-dimethyloct-6-en-1-yl)trifluoroborate having a chemical structure set forth in FIG. 18.

In another embodiment, the present disclosure teaches a synthetic schema for the installation of alpha boronic acids and subsequent trifluoroborates of the Bortezomib family having the chemical structure(s) set forth in FIG. 23.

In another embodiment, the present disclosure teaches methods of synthesizing Bortezomib having a chemical structure set forth in FIG. 24.

In another embodiment, the present disclosure teaches methods of treating cancer(s), immunological disorders, and other diseases in humans.

In another embodiment, the present disclosure teaches methods of treating cancer(s), using Boron Neutron Capture Therapy (“BNCT”) in humans.

In another embodiment, the present disclosure teaches methods of treating cancer(s), using Proton Boron Fusion Therapy (“PBFT”) in humans.

### BRIEF DESCRIPTION OF THE FIGURES

FIG. 1. Conceptual Schema for Installation of Alpha Boronic Acids and Subsequent Trifluoroborates.

FIG. 2. Synthesis of FBH and FBF (L-Phenylalanine Mimetics).

FIG. 3. Purity Analysis of FBH (LCMS Chromatogram).

FIG. 4. Purity Analysis of FBH (LCMS Chromatogram).

FIG. 5. Purity Analysis of FBF (LCMS Chromatogram).

FIG. 6. Purity Analysis of FBF (LCMS Chromatogram).

FIG. 7. Synthesis of LBH and LBF (L-Leucine Mimetics).

FIG. 8. Purity Analysis of LBH (LCMS Chromatogram).

FIG. 9. Purity Analysis of LBH (LCMS Chromatogram).

FIG. 10. Purity Analysis of LBF (LCMS Chromatogram).

FIG. 11. Purity Analysis of LBF (LCMS Chromatogram).

FIG. 12. Synthesis of MBH and MBF (L-Methionine Mimetics).

FIG. 13. Purity Analysis of MBH (LCMS Chromatogram).

FIG. 14. Purity Analysis of MBF (LCMS Chromatogram).

FIG. 15. Synthesis of PBH and PBF (L-Proline Mimetics).

FIG. 16. Purity Analysis of PBH (LCMS Chromatogram).

FIG. 17. Purity Analysis of PBF (LCMS Chromatogram).

FIG. 18. Synthesis of ((1R)-1-amino-3,7-dimethyloct-6-en-1-yl)boronic acid & Potassium ((1R)-1-amino-3,7-dimethyloct-6-en-1-yl)trifluoroborate (Citronella Derivatives).

FIG. 19. Purity Analysis of ((1R)-1-amino-3,7-dimethyloct-6-en-1-yl)boronic acid (LCMS Chromatogram).

FIG. 20. Purity Analysis of Potassium ((1R)-1-amino-3,7-dimethyloct-6-en-1-yl)trifluoroborate (LCMS Chromatogram).

FIG. 21. Evaluation of BPA, PheBF₃ & L-LBH Uptake in FaDu Cells In Vitro. FIG. 21(A). shows the ranking of the uptake of all four compounds in the assay. FIG. 21(B). Shows the concentration of ng Boron/mg Protein at 2 hours. FIG. 21(C). Shows the compound characteristics used in the assay.

FIG. 22. Evaluation of BPA, PheBOH, PheBF₃ & LeuBOH Uptake in FaDu and U343MG Cells In Vitro. FIG. 22(A). Shows the ranking of the uptake of all four compounds in the assay.

FIG. 22(B). Shows the concentration of ng Boron/mg Protein at 2 hours. FIG. 22(C). Shows the compound characteristics used in the assay.

FIG. 23. Synthesis Scheme of the Bortezomib Family (Bortezomib, Delanzomib, and Ixazomib.

FIG. 24. Synthesis of Bortezomib.

FIG. 25. Purity Analysis of Bortezomib (LCMS Chromatogram).

### DETAILED DESCRIPTION OF THE INVENTION

### Outline of Sections

I.) Definitions
II.) BPA
III.) BSH
IV.) Boron
   a. Boron Generally
V.) Naturally Occurring Amino Acids
VI.) Trifluoroborate Compounds With the installation of Alpha Boronic Acids
   a. L-Phenylalanine Mimetics
      i. FBH
      ii. FBF
   b. L-Leucine Mimetics
      i. LBH
      ii. LBF
   c. L-Methionine Mimetics
      i. MBH
      ii. MBF
   d. L-Proline Mimetics
      i. PBH
      ii. PBF
   e. Citronella Derivatives
      i. ((1R)-1-amino-3,7-dimethyloct-6-en-1-yl)boronic acid
      ii. Potassium ((1R)-1-amino-3,7-dimethyloct-6-en-1-yl)trifluoroborate
   f. Bortezomib Family
      i. Bortezomib
      ii. Delanzomib
      iii. Ixazomib
VII.) Boron Neutron Capture Therapy Using BF₃ Compounds
VIII.) Proton Boron Fusion Therapy Using BF₃ Compounds
IX.) Methods of Delivering BF₃ Compounds to a Cell
X.) KITS/Articles of Manufacture
XI.) Treatment of Cancer(s) using BF₃ Compounds
XII.) Treatment of Cancer(s) using BF₃ Compound Cocktails
XIII.) Combination Therapy

### I.) Definitions

Unless otherwise defined, all terms of art, notations and other scientific terms or terminology used herein are intended to have the meanings commonly understood by those of skill in the art to which this invention pertains unless the context clearly indicates otherwise. In some cases, terms with commonly understood meanings are defined herein for clarity and/or for ready reference, and the inclusion of such definitions herein should not necessarily be construed to represent a substantial difference over what is generally understood in the art.

When a trade name is used herein, reference to the trade name also refers to the product formulation, the generic drug, and the active pharmaceutical ingredient(s) of the trade name product, unless otherwise indicated by context.

The terms “advanced cancer”, “locally advanced cancer”, “advanced disease” and “locally advanced disease” mean cancers that have extended through the relevant tissue capsule and are meant to include stage C disease under the American Urological Association (AUA) system, stage C1-C2 disease under the Whitmore-Jewett system, and stage T3-T4 and N+ disease under the TNM (tumor, node, metastasis) system. In general, surgery is not recommended for patients with locally advanced disease and these patients have substantially less favorable outcomes compared to patients having clinically localized (organ-confined) cancer.

“Amino Acid” means a simple organic compound containing both a carboxyl (—COOH) and an amino (—NH₂) group.

“Boronic Acid” means an organic compound related to boric acid (B(OH)₃) in which one of the three hydroxyl groups (—OH) is replaced by an alkyl or aryl group (represented by R in the general formula R—B(OH)₂). As a compound containing a carbon-boron bond, members of this class thus belong to the larger class of organoboranes.

“Borylation” means reactions that produce an organoboron compound through functionalization of aliphatic and aromatic C—H bonds.

“Organotrifluoroborate” or “trifluoroborate” (BF₃) means organoboron compounds that contain an anion with the general formula [RBF₃]—. They can be thought of as protected boronic acids, or as adducts of carbanions and boron trifluoride. Organotrifluoroborates are tolerant of air and moisture and are easy to handle and purify. They are often used in organic synthesis as alternatives to boronic acids (RB(OH)₂), boronate esters (RB(OR′)₂), and organoboranes (R₃B), particularly for Suzuki-Miyaura coupling.

The term “compound” refers to and encompasses the trifluoroborate chemical compound(s) (e.g., a BF₃) with the installation of boronic acids, as well as, whether explicitly stated or not, and unless the context makes clear that the following are to be excluded: amorphous and crystalline forms of the compound, including polymorphic forms, where these forms may be part of a mixture or in isolation; free acid and free base forms of the compound, which are typically the forms shown in the structures provided herein; isomers of the compound, which refers to optical isomers, and tautomeric isomers, where optical isomers include enantiomers and diastereomers, chiral isomers and non-chiral isomers, and the optical isomers include isolated optical isomers as well as mixtures of optical isomers including racemic and non-racemic mixtures; where an isomer may be in isolated form or in a mixture with one or more other isomers; isotopes of the compound, including deuterium- and tritium-containing compounds, and including compounds containing radioisotopes, including therapeutically- and diagnostically-effective radioisotopes; multimeric forms of the compound, including dimeric, trimeric, etc. forms; salts of the compound, preferably pharmaceutically acceptable salts, including acid addition salts and base addition salts, including salts having organic counterions and inorganic counterions, and including zwitterionic forms, where if a compound is associated with two or more counterions, the two or more counterions may be the same or different; and solvates of the compound, including hemisolvates, monosolvates, disolvates, etc., including organic solvates and inorganic solvates, said inorganic solvates including hydrates; where if a compound is associated with two or more solvent molecules, the two or more solvent molecules may be the same or different. In some instances, reference made herein to a compound of the invention will include an explicit reference to one or of the above forms, e.g., salts and/or solvates; however, this reference is for emphasis only, and is not to be construed as excluding other of the above forms as identified above

The terms “inhibit” or “inhibition of” as used herein means to reduce by a measurable amount, or to prevent entirely.

The term “mammal” refers to any organism classified as a mammal, including mice, rats, rabbits, dogs, cats, cows, horses, and humans. In one embodiment of the invention, the mammal is a mouse. In another embodiment of the invention, the mammal is a human.

The terms “metastatic cancer” and “metastatic disease” mean cancers that have spread to regional lymph nodes or to distant sites and are meant to include stage D disease under the AUA system and stage T×N×M+ under the TNM system.

“Molecular recognition” means a chemical event in which a host molecule is able to form a complex with a second molecule (i.e., the guest). This process occurs through non-covalent chemical bonds, including but not limited to, hydrogen bonding, hydrophobic interactions, ionic interaction.

“Pharmaceutically acceptable” refers to a non-toxic, inert, and/or composition that is physiologically compatible with humans or other mammals.

The term “neutron capture agent” means a stable non-reactive chemical isotope which, when activated by neutrons produces alpha particles.

The term “neutron capture therapy” means a noninvasive therapeutic modality for treating locally invasive malignant tumors such as primary brain tumors and recurrent head and neck cancer and other immunological disorders and disease by irradiating a neutron capture agent with neutrons.

The term “Suzuki-Miyaura reaction” or “Suzuki coupling” means an organic reaction, classified as a cross-coupling reaction, where the coupling partners are a boronic acid and an organohalide and the catalyst is a palladium(0) complex. It was first published in 1979 by Akira Suzuki. It is widely used to synthesize polyolefins, styrenes, and substituted biphenyls. The general scheme for the Suzuki reaction is shown below:

$${R}^{1} - X + {R}^{2} - {BY}_{2} {⟶}^{[ Pd ] cat . , base} {R}^{1} - {R}^{2}$$

where a carbon-carbon single bond is formed by coupling a halide (R¹—X) with an organoboron species (R²—BY₂) using a palladium catalyst and a base.

As used herein “to treat” or “therapeutic” and grammatically related terms, refer to any improvement of any consequence of disease, such as prolonged survival, less morbidity, and/or a lessening of side effects which are the byproducts of an alternative therapeutic modality; as is readily appreciated in the art, full eradication of disease is a preferred but albeit not a requirement for a treatment act.

### II.) BPA

By way of reference, (¹⁰B)-BPA, L-BPA, or 4-Borono-L-phenylalanine (Sigma Aldrich, St. Louis, MO) is a synthetic compound with the chemical formula:

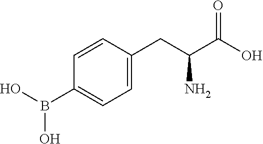 [SMILES: N[C@@H](Cc1ccc(B(O)O)cc1)C(=O)O]

and is an important boronated compound useful in the treatment of cancer though BNCT. It is a widely known compound which many syntheses have been developed (See, U.S. Pat. No. 8,765,997, Taiwan Biotech Co, Ltd., Taoyuan Hsein, Taiwan, and US2017/0015684, Stella Pharma Corp., Osaka Prefecture Univ., Osaka, Japan)

### III.) BSH

In addition, BSH, or sodium borocaptate, or BSH sodium borocaptate, or Borocaptate sodium B10, or un-decahydrododecaborane thiol is a synthetic chemical compound with the chemical formula:

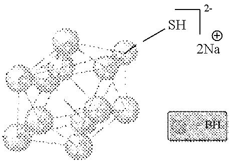

where boron atoms are represented by dots in the vertices for the ecosahedron. BSH is used as a capture agent in BNCT. Generally speaking, BSH is injected into a vein and becomes concentrated in tumor cells. The patient then receives radiation treatment with atomic particles called neutrons. The neutrons fuse with the boron nuclei in BSH and to produce high energy alpha particles that kill the tumor cells.

### IV.) Boron

### (a.) Boron Generally

Generally speaking, and for purposes of this disclosure, Boron is a chemical element with symbol B and atomic number five (5). Primarily used in chemical compounds, natural boron is composed of two stable isotopes, once of which is Boron-10 and the other is Boron-11. Boron-10 isotope is useful for capturing epithermal neutrons, which makes it a promising tool in a therapeutic context using Boron Neutron Capture Therapy. Biologically, the borylated compounds disclosed herein are nontoxic to humans and animals. Based on the foregoing, it will be readily apparent to one of skill in the art that improved modalities for providing high concentrations of boron into a cancer cell are advantageous. It is an object of the present disclosure to provide that advantage.

### V.) Naturally Occurring Amino Acids

Generally speaking, and for the purposes of this disclosure, naturally occurring amino acids are organic compounds containing amine (—NH₂) and carboxyl (—COOH) functional groups, along with a side chain (R group) specific to each amino acid. The key elements of an amino acid are carbon (C), hydrogen (H), oxygen (O), and nitrogen (N), although other elements are found in the side chains of certain amino acids. About 500 naturally occurring amino acids are known (though only 20 appear in the genetic code (Table 1)) and can be classified in many ways. They can be classified according to the core structural functional groups' locations as alpha- (α-), beta- (β-), gamma- (γ-) or delta- (δ-) amino acids; other categories relate to polarity, pH level, and side chain group type (aliphatic, acyclic, aromatic, containing hydroxyl or sulfur, etc.). In the form of proteins, amino acid residues form the second-largest component (water is the largest) of human muscles and other tissues. Beyond their role as residues in proteins, amino acids participate in a number of processes such as neurotransmitter transport and biosynthesis.

The twenty (20) amino acids encoded directly by the genetic code (See, Table 1) can be divided into several groups based on their properties. Principal factors are charge, hydrophilicity or hydrophobicity, size, and functional groups. These properties are important for protein structure and protein-protein interactions. The water-soluble proteins tend to have their hydrophobic residues (Leu, lie, Val, Phe, and Trp) buried in the middle of the protein, whereas hydrophilic side chains are exposed to the aqueous solvent.

The integral membrane proteins tend to have outer rings of exposed hydrophobic amino acids that anchor them into the lipid bilayer. In the case part-way between these two extremes, some peripheral membrane proteins have a patch of hydrophobic amino acids on their surface that locks onto the membrane. In similar fashion, proteins that have to bind to positively charged molecules have surfaces rich with negatively charged amino acids like glutamate and aspartate, while proteins binding to negatively charged molecules have surfaces rich with positively charged chains like lysine and arginine. There are different hydrophobicity scales of amino acid residues.

Some amino acids have special properties such as cysteine, which can form covalent disulfide bonds to other cysteine residues, proline that forms a cycle to the polypeptide backbone, and glycine that is more flexible than other amino acids.

### VI.) Trifluoroborate Compounds Utilizing the Installation of Alpha Boronic Acids

Aminoboronic acids have played a key role in both the pharmaceutical industry and medicinal chemistry. In fact, the first proteasome inhibitor drug to be tested in humans contains such a motif and later became known as Bortezomib, or VELCADE by Millenium Pharmaceuticals. The boronic acid in Bortezomib stems from the boroleucine unit in the modified dipeptide and is responsible for the inhibitory effect of the drug. The designed synthesis utilizes a convergent approach whereby each of the three synthons (pyrazinecarboxylic acid, L-phenylalanine, and L-boroleucine) are coupled via a peptide bond in a subsequent fashion.

A significant portion of the synthesis revolves around the boroleucine moiety since aminoboronic acids are non-naturally occurring. Due to this, fabrication of such moieties requires chiral auxiliaries and harsh conditions to install the boronic acid in an enantiomeric manner. In the beginning of the synthesis, isobutylboronic acid was used in conjunction with a chiral diol auxiliary, (S)-(+)-pinanediol, to form a boronate ester. See, WO2005/097809, Millenium Pharma, Inc. Subsequent homologation was achieved via Matteson's conditions where the lithium salt of methylene chloride forms a chiral α,α-dichloroboronate complex and rapidly undergoes a stereoselective internal rearrangement, ultimately forming a chiral α-chloroboronic ester. See, for example, MATTESON, et. al., Ann. Chem. 1195 (1995). Notably, it was then discovered that the addition of zinc (II) chloride improved this transformation via catalysis. The installation of the amine is achieved via lithium bis(trimethylsilyl)amide (LiHMDS) followed by desilylation, affording the aminoboronic ester.

To circumvent the expensive costs of using the chiral auxiliary, it was later determined that the achiral diol could be used, and a down-stream classical resolution could be implemented to separate the stereoisomers. See, MATTESON, et. al., Organometallics 2(11), pp. 1529-1535 (1983). By taking advantage of the naturally occurring chiral L-phenylalanine synthon that is found in Bortezomib, those of skill in the art were able to selectively isolate the desired diastereomer post-peptide coupling via either crystallization, chromatography or stereoselective hydrolysis. After isolating the desired diastereomer, subsequent peptide coupling with the pyrazinecarboxylic acid afforded Bortezomib as the boronate ester. Deprotection of the boronate ester was feasible via transesterification in the presence of isobutylboronic acid, yielding Bortezomib as the product.

Other syntheses have been investigated since the discovery of Bortezomib. Many of which utilized Ellman's sulfinamide ((R) or (S) tert-butyl sulfinamide) to enantiomerically install a boronate ester for the boroleucine synthon. See, CN101781326. Notably, XIN, et a, described a method where they performed an amine condensation of Ellman's sulfinamide onto isovaleraldehyde to yield the corresponding sulfinimine. Id. Using (1,3-dicyclohexylimidazol-2-ylidene)copper(I) tert-butoxide as a catalyst along with a chiral boronate ester, α-borylation to the sulfinimine was achievable. The product underwent sulfinamide bond cleavage in the presence of hydrochloric acid to afford the amino borate as described in the art. See, ELLMAN, et al., Am. Chem. Soc. 130(22), 6910-6911 (2008). From there, they synthesized Bortezomib as previously discussed by PICKERSGILL, et al., supra.

Implementing Ellman's sulfinamide, a copper catalyst, and a chiral boronate ultimately allows one to install an α-boronate without the use of harsh, inert conditions such as those used by Matteson's homologation. However, using a chiral boronate is still commercially prohibitive to use at a production level and chiral resolution of a racemate inevitably diminishes yields.

Based on the foregoing, this disclosure teaches that by combining methodologies, an inexpensive more efficient route towards aminoboronic acids can be achieved. By taking the chiral Ellman's sulfinamide ((R)-tert-butyl sulfinamide) in conjunction with isovaleraldehyde and performing an amine condensation, a more commercially viable chiral environment can be introduced without the need of a more commercially prohibitive chiral boronate. Using the chiral sulfinamide instead of the racemate and later performing a chiral resolution improves the overall yield by avoiding the unwanted enantiomer.

Borylation alpha to the corresponding sulfinimine in the presence of copper (II) sulfate pentahydrate (CuSO₄·5H₂O), tricyclohexylphosphonium tetrafluoroborate (Cy₃PHBF₄), and bis(pinacolato)diboron (B₂pin₂), is observed as previously reported. See, ELLMAN, et al., J. Org. Chem., 79 pp. 3671-3677 (2014). The corresponding boronate undergoes a global deprotection where both the sulfinamide bond and the boronate ester are cleaved in the presence of hydrochloric acid and cool conditions, yielding the boroleucine as a hydrogen chloride salt.

This novel method takes advantage of low reagent costs, more process-friendly conditions, and great atom economy. Originally, the synthesis takes four (4) steps to yield the aminoborate whereas this methodology permits the isolation of the aminoboronic acid in only three (3) steps. As a result, this ultimately shortens the synthesis of Bortezomib by two (2) steps since transesterification would no longer be warranted. Incorporating multiple routes with a novel global deprotection step ultimately saves the manufacturer's time and production costs in synthesizing pharmaceutical drugs which could then be reflected in the costs for patients.

The method disclosed herein can be used in a myriad of ways creating numerous BF₃ compounds.

Based on the aforementioned rationale, as set forth in this disclosure are trifluoroborate (BF₃) compounds that were evaluated for in vitro uptake using various cancer cell lines. Additionally, it is taught in this disclosure that established sub-cutaneous xenografts models in mice were used to determine pharmacokinetics and biodistribution of boron in a tumor, blood, and other organs.

The therapeutic potential of BNCT rests in the selective accumulation of a sufficient amount of ¹⁰B within cancer cells. To investigate the ability of a new class of tribluoroborate compounds to be used as boron capture agents, the disclosure teaches numerous synthesized BF₃ compounds interrogated at multiple ranges of concentrations illustrating what is believed to be the physiologically relevant amounts for boronophenylalanine (BPA), currently the most widely studied boron drug in BNCT clinical practice.

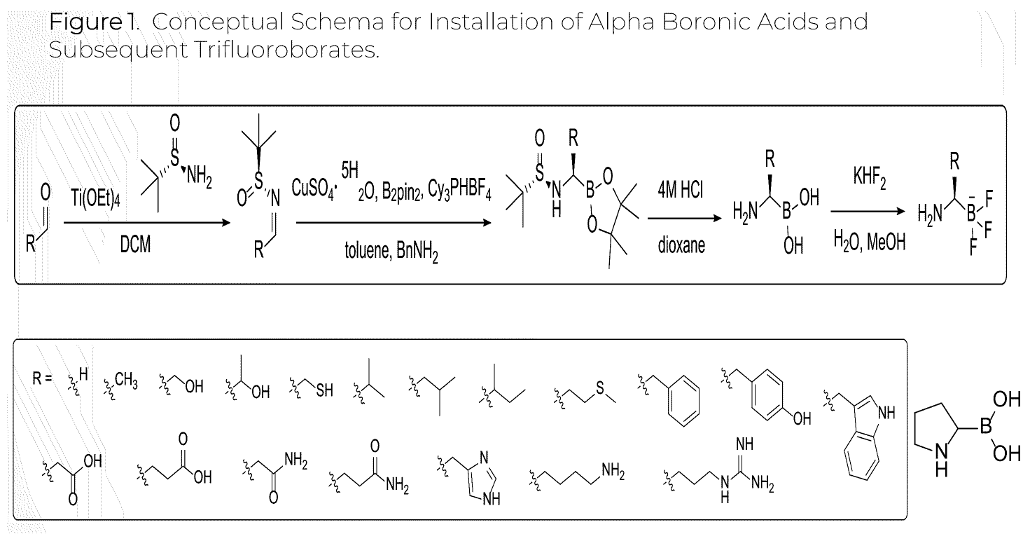

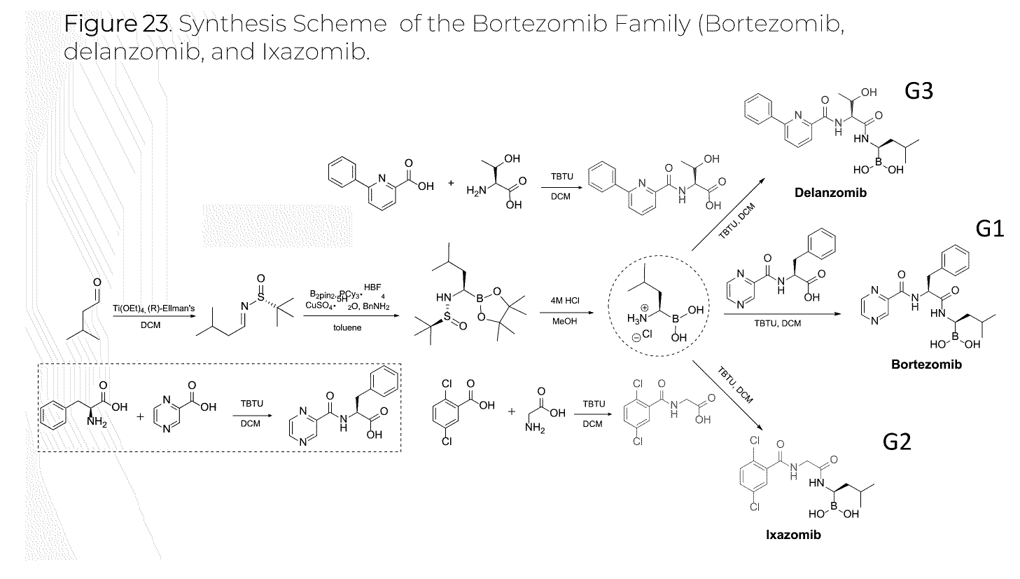

Based on the above-referenced background and therapeutic rationale, the following trifluoroborate (BF₃) compounds are disclosed herein and more fully referenced in FIG. 1 and FIG. 23. Specific characterization of the chemical structures is more fully set forth below.

### (a) L-Phenylalanine Mimetics

Using the conceptual schema for the installation of alpha boronic acids set forth in FIG. 1, the present disclosure teaches the following BF₃ compounds using L-phenylalanine mimetics.

### (i) FBH

In one embodiment, a composition with the following formula is within the scope of the of the present disclosure:

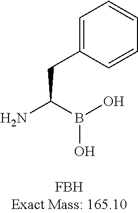 [SMILES: N[C@@H](Cc1ccccc1)B(O)O]

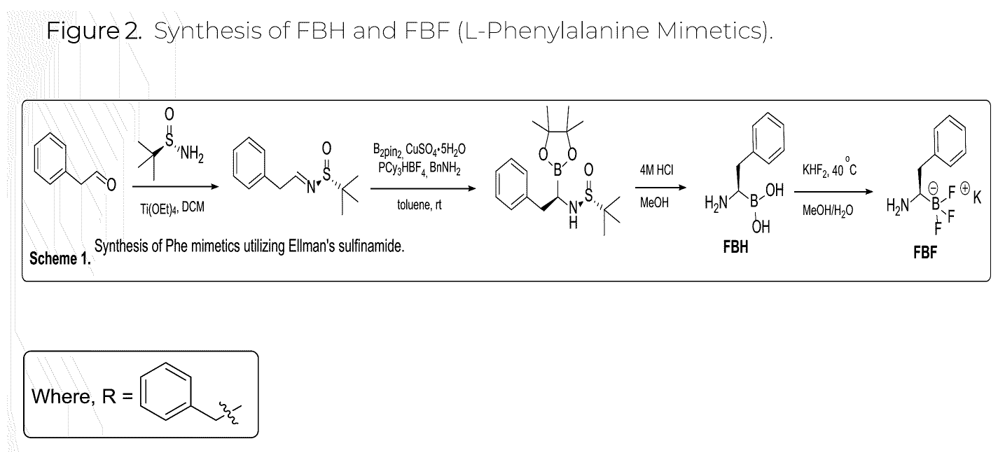

It will be appreciated by one of ordinary skill in the art that the above composition is synthesized as shown in FIG. 2.

### (ii) FBF

In a further embodiment, a composition with the following formula is within the scope of the of the present disclosure:

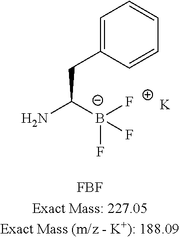 [SMILES: N[C@@H](Cc1ccccc1)[B-](F)(F)F ; [K+]]

It will be appreciated by one of ordinary skill in the art that the above composition is synthesized as shown in FIG. 2.

### (b) L-Leucine Mimetics

Using the conceptual schema for the installation of alpha boronic acids set forth in FIG. 1, the present disclosure teaches the following BF₃ compounds using L-leucine mimetics.

### (i) LBH

In one embodiment, a composition with the following formula is within the scope of the of the present disclosure:

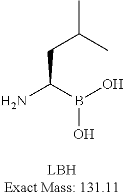

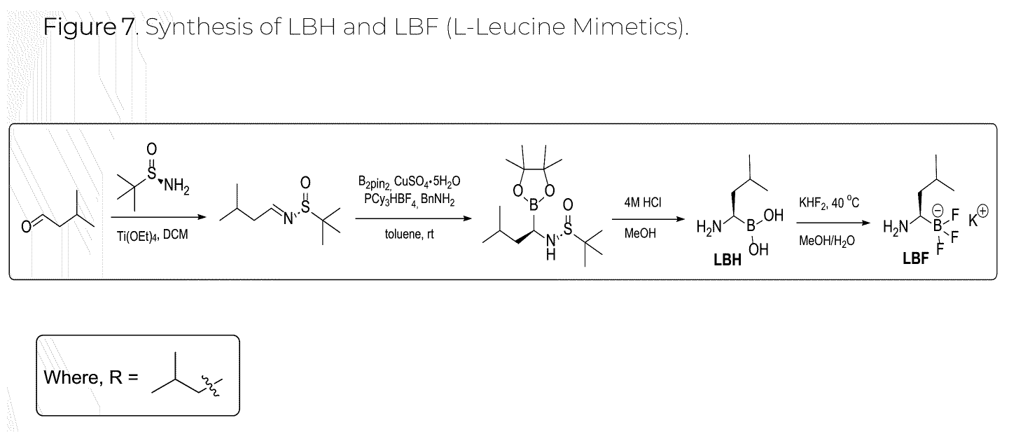

It will be appreciated by one of ordinary skill in the art that the above composition is synthesized as shown in FIG. 7.

### (ii) LBF

In a further embodiment, a composition with the following formula is within the scope of the of the present disclosure:

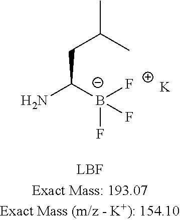

It will be appreciated by one of ordinary skill in the art that the above composition is synthesized as shown in FIG. 7.

### (c) L-Methionine Mimetics

Using the conceptual schema for the installation of alpha boronic acids set forth in FIG. 1, the present disclosure teaches the following BF₃ compounds using L-Methionine mimetics.

### (i) MBH

In one embodiment, a composition with the following formula is within the scope of the of the present disclosure:

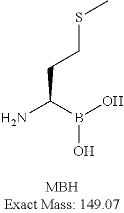

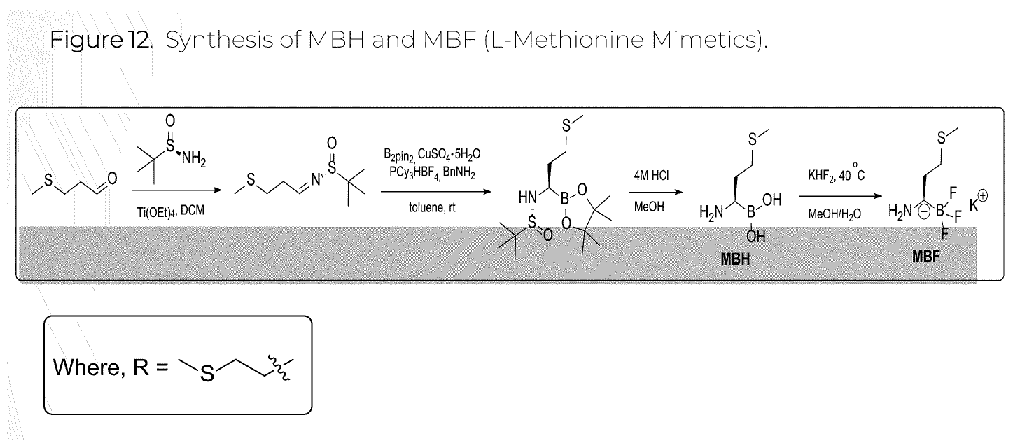

It will be appreciated by one of ordinary skill in the art that the above composition is synthesized as shown in FIG. 12.

### (ii) MBF

In a further embodiment, a composition with the following formula is within the scope of the of the present disclosure:

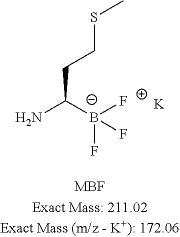

It will be appreciated by one of ordinary skill in the art that the above composition is synthesized as shown in FIG. 12.

### (d) L-Proline Mimetics

Using the conceptual schema for the installation of alpha boronic acids set forth in FIG. 1, the present disclosure teaches the following BF₃ compounds using L-Proline mimetics.

### (i) PBH

In one embodiment, a composition with the following formula is within the scope of the of the present disclosure:

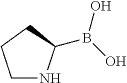

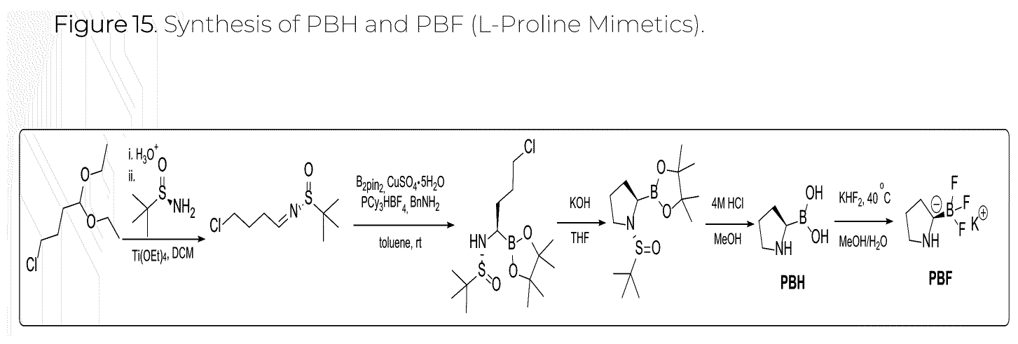

It will be appreciated by one of ordinary skill in the art that the above composition is synthesized as shown in FIG. 15.

### (ii) PBF

In a further embodiment, a composition with the following formula is within the scope of the of the present disclosure:

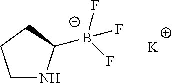

It will be appreciated by one of ordinary skill in the art that the above composition is synthesized as shown in FIG. 15.

### (e) Citronella Derivative(s)

Using the conceptual schema for the installation of alpha boronic acids set forth in FIG. 1, the present disclosure teaches the following BF₃ compounds.

### (i) ((1R)-1-amino-3,7-dimethyloct-6-en-1-yl)boronic acid

In one embodiment, a composition with the following formula is within the scope of the of the present disclosure:

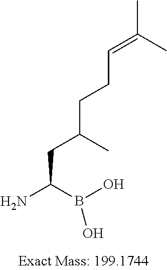

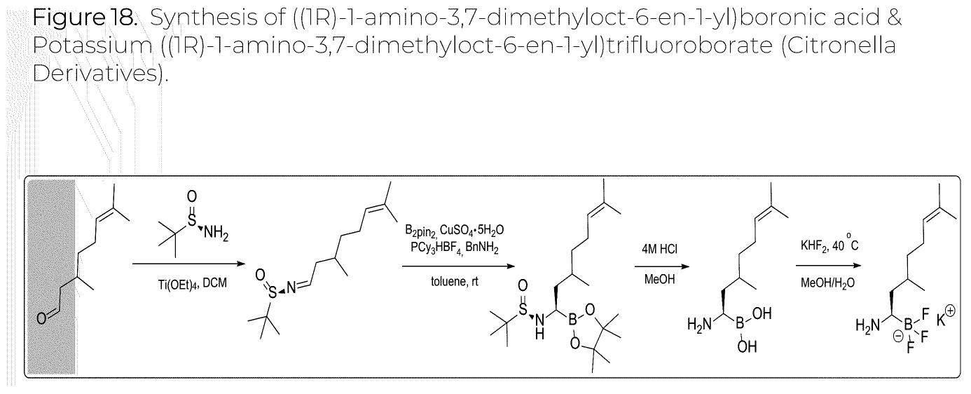

It will be appreciated by one of ordinary skill in the art that the above composition is synthesized as shown in FIG. 18.

### (ii) Potassium ((1R)-1-amino-3,7-dimethyloct-6-en-1-yl)trifluoroborate

In a further embodiment, a composition with the following formula is within the scope of the of the present disclosure:

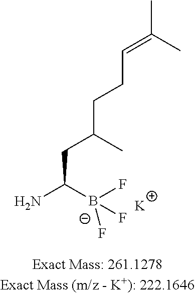

It will be appreciated by one of ordinary skill in the art that the above composition is synthesized as shown in FIG. 18.

### (f) Bortezomib Family

Using the conceptual schema for the installation of alpha boronic acids set forth in FIG. 1, the application of the present disclosure teaches the following BF₃ compounds used to synthesize members of the Bortezomib family using the synthesis schema set forth in FIG. 23.

### (i) Bortezomib

In one embodiment, a composition with the following formula is within the scope of the of the present disclosure:

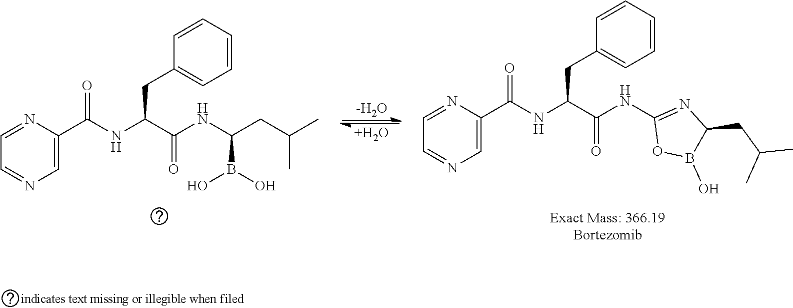

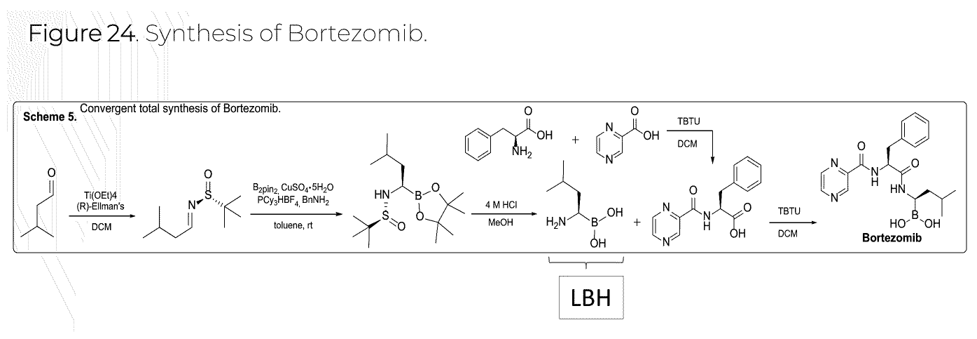

It will be appreciated by one of ordinary skill in the art that the above composition is synthesized as shown in FIG. 24.

### (ii) Delanzomib

In one embodiment, a composition with the following formula is within the scope of the of the present disclosure:

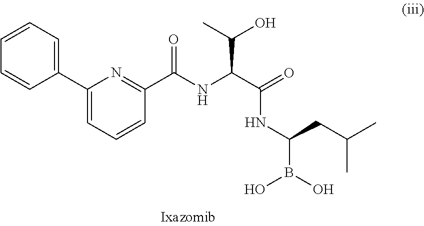 [SMILES: CC(C)C[C@H](NC(=O)[C@@H](NC(=O)c1cccc(-c2ccccc2)n1)C(C)O)B(O)O]

In one embodiment, a composition with the following formula is within the scope of the of the present disclosure:

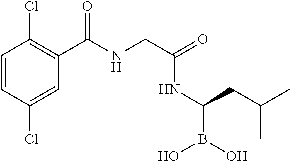 [SMILES: CC(C)C[C@H](NC(=O)CNC(=O)c1cc(Cl)ccc1Cl)B(O)O]

It will be appreciated by one of ordinary skill in the art that the synthesis of the BF₃ compounds of the present disclosure, have a functional uptake in certain complexes can be synthesized to deliver concentrated amounts of the BF₃ compounds to a cancer or otherwise diseased cell for use in BNCT and/or other cancer treatment modalities. Based on the ability for certain antigen complexes to upregulate specific amino acids, various BF₃ compounds can be explored. For purposes of this disclosure, in one embodiment, the amino acid comprises valine, methionine, leucine, proline, isoleucine, histidine, tryptophan, tyrosine, and any amino acid set forth in Table I.

The wide diversity of useful reactivity that is specific to the BF₃ synthesis schema and subsequent compounds is contemplated herein and is readily understood by one of ordinary skill in the art. Subsequent to the synthesis of the compounds disclosed herein, additional antigen complexes and transporters may be implicated through selective interrogation of BF₃ compounds and their respective molecular substrates.

### VII.) Boron Neutron Capture Therapy Using BF₃ Compounds

One aspect of the present disclosure is the use of BF₃ compounds as a modality for Boron Neutron Capture Therapy (BNCT) and/or Boron Proton Capture Therapy (“BPCT”). Briefly, BNCT is a binary treatment modality in which neither component alone is lethal or toxic to the tumor. The two components comprise (i) the infusion or delivery of a capture compound, which preferentially is concentrated in the tumor, and (ii) the irradiation of the tumor site by neutrons or by protons. In BNCT, given the large cross-section of thermal neutron interactions with ¹⁰B, there is consequently a high probability of a splitting of Boron nucleus into ⁴He²⁺ and ⁷Li⁺. Given that the ionization capability of He²⁺ and Li⁺ is high, and the distances travelled are short, then the cells preferably enriched by Boron are killed and the healthy cells are damaged much less due to the lack of high concentration of boron. Given this, the advantage of BNCT is the destruction of tumor cells without a highly traumatic surgical procedure. However, as will be understood by one of skill in the art, success is predicated high concentration and selective localization of ¹⁰B in tumor cells.

In one embodiment, a BF₃ compound is used in lieu of ¹⁰B and is shown to have better properties of concentration and uptake into a tumor cell and is thus contemplated as a neutron capture agent. The BF₃ compound is then given to a patient and the BF₃ compound is localized into a tumor cell. The BF₃ compounds are then concentrated into the tumor and the tumor is irradiated using epithermal neutrons. The tumor cells are destroyed.

### VIII. Proton Boron Fusion Therapy Using BF₃ Compounds

Another aspect of the present disclosure is the use of BF₃ as a modality for Proton Boron Fusion Therapy (PBFT). Briefly, the proton boron fusion reaction was introduced in the 1960s. Three alpha particles are emitted after the reaction between a proton (¹H) and a boron particle (¹¹B). These three alpha particles provide the damage to the tumor cell, just as in the case of alpha particles in BNCT. Theoretically, in the case of PBFT, the therapy efficacy per incident particle is three times (3×) greater than that of BNCT. In addition, because the proton beam has the advantage of a Bragg-peak characteristic, normal tissue damage can be reduced. Generally speaking, many studies for tumor treatment using alpha particles have been performed. In order to take advantage of alpha particles for dose delivery, two key points should be considered. First, the boron uptake should be labeled accurately to the target cell. As mentioned previously, alpha particles are generated where the boronated compound is accumulated. If this happens in normal tissue near the tumor region, alpha particles will damage the normal tissue as well as the tumor cell. Second, the number of generated alpha particles is also a significant factor for effective therapy. By using PBFT, a more effective therapy can be realized compared to BNCT or conventional proton therapy alone.

In one embodiment, a BF₃ compound of the present disclosure is synthesized using the schema(s) and methods disclosed herein. The BF₃ compound is then given to a patient and the BF₃ compound is localized into a tumor cell. The BF₃ compound is concentrated into the tumor and the tumor is irradiated using epithermal neutrons. The tumor cells are destroyed.

### IX. Methods of Delivering BF₃ Compounds to a Cell

As will be appreciated by one of ordinary skill in the art, the ability to efficiently deliver high concentrations of BF₃ compounds to a cell is an advantage of the present invention.

It is shown that the BF₃ compounds of the present disclosure enables a higher amount of active capture agent versus boron to be administered to a cell safely in mammals. Briefly, BF₃ compounds of the disclosure are prepared as set forth in the disclosure. The resulting BF₃ compounds are taken up by the tumor cell by the upregulated LAT1 transporter protein and/or an upregulated dipeptide transporter protein such as PEPT1.

### X.) Kits/Articles of Manufacture

For use in the laboratory, prognostic, prophylactic, diagnostic and therapeutic applications described herein, kits are within the scope of the invention. Such kits can comprise a carrier, package, or container that is compartmentalized to receive one or more containers such as vials, tubes, and the like, each of the container(s) comprising one of the separate elements to be used in the method, along with a label or insert comprising instructions for use, such as a use described herein. For example, the container(s) can comprise a BF₃ compound or several BF₃ compounds of the disclosure. Kits can comprise a container comprising a drug unit. The kit can include all or part of the BF₃ compounds and/or diagnostic assays for detecting cancer and/or other immunological disorders.

The kit of the invention will typically comprise the container described above, and one or more other containers associated therewith that comprise materials desirable from a commercial and user standpoint, including buffers, diluents, filters, needles, syringes; carrier, package, container, vial and/or tube labels listing contents and/or instructions for use, and package inserts with instructions for use.

A label can be present on or with the container to indicate that the composition is used for a specific therapy or non-therapeutic application, such as a prognostic, prophylactic, diagnostic or laboratory application, and can also indicate directions for either in vivo or in vitro use, such as those described herein. Directions and or other information can also be included on an insert(s) or label(s) which is included with or on the kit. The label can be on or associated with the container. A label can be on a container when letters, numbers or other characters forming the label are molded or etched into the container itself; a label can be associated with a container when it is present within a receptacle or carrier that also holds the container, e.g., as a package insert. The label can indicate that the composition is used for diagnosing, treating, prophylaxing or prognosing a condition, such as a cancer or other immunological disorder.

The terms “kit” and “article of manufacture” can be used as synonyms.

In another embodiment of the invention, an article(s) of manufacture containing compositions, such as BF₃ compounds of the disclosure. The article of manufacture typically comprises at least one container and at least one label. Suitable containers include, for example, bottles, vials, syringes, and test tubes. The containers can be formed from a variety of materials such as glass, metal, or plastic. The container can hold one or several BF₃ compounds and/or one or more therapeutics doses of BF₃ compounds.

The container can alternatively hold a composition that is effective for treating, diagnosis, prognosing or prophylaxing a condition and can have a sterile access port (for example the container can be an intravenous solution bag or a vial having a stopper pierceable by a hypodermic injection needle). The active agents in the composition can be a BF₃ compounds of the present disclosure.

The article of manufacture can further comprise a second container comprising a pharmaceutically acceptable buffer, such as phosphate-buffered saline, Ringer's solution, and/or dextrose solution. It can further include other materials desirable from a commercial and user standpoint, including other buffers, diluents, filters, stirrers, needles, syringes, and/or package inserts with indications and/or instructions for use.

### XI.) Treatment of Cancer(s) Using BF₃ Compounds

The identification of BF₃ compounds synthesized using the approaches described in the disclosure opens a number of therapeutic approaches to the treatment of cancers, in addition to a plurality of other immunological disorders.

Of note, targeted antitumor therapies have been useful even when the targeted protein is expressed on normal tissues, even vital normal organ tissues. A vital organ is one that is necessary to sustain life, such as the heart or colon. A non-vital organ is one that can be removed whereupon the individual is still able to survive. Examples of non-vital organs are ovary, breast, and prostate. Expression of a target protein in normal tissue, even vital normal tissue, does not defeat the utility of a BF₃ compound as a therapeutic for certain tumors. In addition, some vital organs are not affected by normal organ expression because of an immunoprivilege. Immunoprivileged organs are organs that are protected from blood by a blood-organ barrier and thus are not accessible to immunotherapy. Examples of immunoprivileged organs are the brain and testis.

Accordingly, therapeutic approaches that use BF₃ compounds to inhibit the activity of a cancer related protein are useful for patients suffering from those cancer(s). These therapeutic approaches generally fall into three classes. The first class is where a BF₃ compound modulates the function as it relates to tumor cell growth leading to inhibition or retardation of tumor cell growth or inducing its killing. The second class comprises using a BF₃ compound for inhibiting the binding or association of a cancer-related protein with its binding partner or with other proteins. The third class comprises a variety of methods of using BF₃ compounds for inhibiting the transcription of a cancer-related gene or translation of a cancer related mRNA.

Accordingly, cancer patients can be evaluated for the presence and level of cancer-related protein expression, preferably using immunohistochemical assessments of tumor tissue, quantitative imaging, or other techniques that reliably indicate the presence and degree of cancer-related expression. Immunohistochemical analysis of tumor biopsies or surgical specimens is preferred for this purpose. Methods for immunohistochemical analysis of tumor tissues are well known in the art. Once the proper cancer-related protein is determined, then the appropriate BF₃ compound is used as a sole agent, or in combination to treat the cancer.

### XII.) Treatment of Cancer(s) Using BF₃ Compound Cocktails

Therapeutic methods of the invention contemplate the administration of single BF₃ compound(s) as well as combinations, or cocktails, of BF₃ compound(s) (e.g., BF₃ compound(s) that are directed to a different cancer-related protein or receptor). Such BF₃ compound cocktails can have certain advantages inasmuch as they contain BF₃ compound that target different epitopes, exploit different effector mechanisms, or combine directly cytotoxic BF₃ compound(s) with BF₃ compounds that rely on immune effector functionality, Such BF₃ compound(s) in combination can exhibit synergistic therapeutic effects. In addition, BF₃ compound(s) can be administered concomitantly with other therapeutic modalities, including but not limited to various chemotherapeutic and biologic agents, androgen-blockers, immune modulators (e.g., IL-2, GM-CSF), surgery, radiation, BNCT, and or Proton Boron Fusion Therapy. In a preferred embodiment, the BF₃ compound(s) are administered in combination form.

BF₃ compound formulations are administered via any route capable of delivering the BF₃ compound(s) to a tumor cell. Routes of administration include, but are not limited to, intravenous, intraperitoneal, intramuscular, intratumor, intradermal, and the like. Treatment generally involves repeated administration of the BF₃ compound preparation, via an acceptable route of administration such as intravenous injection (IV), typically at a dose in the range, including but not limited to, 0.1, 0.2, 0.3, 0.4, 0.5, 0.6, 0.7, 0.8, 0.9, 1, 2, 3, 4, 5, 6, 7, 8, 9, 10, 15, 20, or 25 mg % kg body weight. In general, doses in the range of 10-1000 mg BF₃ compound per week are effective and well tolerated.

Preferably, the initial loading dose is administered as a 90-minute or longer infusion. The periodic maintenance dose is administered as a 30 minute or longer infusion, provided the initial dose was well tolerated. As appreciated by those of skill in the art, several factors can influence the ideal dose regimen in a particular case. Such factors include, for example, the potency of the BF₃ compound, the degree of the cancer-related protein expression in the patient, the frequency of treatment, and the influence of chemotherapeutic or other agents used in combination with the treatment method of the invention, as well as the health status of a particular patient.

Optionally, patients should be evaluated for the levels of BF₃ compound in a given sample in order to assist in the determination of the most effective dosing regimen, etc. Such evaluations are also used for monitoring purposes throughout therapy and are useful to gauge therapeutic success in combination with the evaluation of other parameters (for example, urine cytology and/or ImmunoCyt levels in bladder cancer therapy, or by analogy, serum PSA levels in prostate cancer therapy).

An object of the present invention is to provide BF₃ compound(s), which inhibit or retard the growth of tumor cells expressing a cancer-related protein. A further object of this invention is to provide methods to inhibit angiogenesis and other biological functions and thereby reduce tumor growth in mammals, preferably humans, using such BF₃ compound(s), and in particular using such BF₃ compound(s) combined with other drugs or immunologically active treatments.

### XIII.) Combination Therapy

In one embodiment, there is synergy when tumors, including human tumors, are treated with BF₃ compound(s) in conjunction with chemotherapeutic agents or radiation or combinations thereof.

In other words, the inhibition of tumor growth by a BF₃ compound is enhanced more than expected when combined with chemotherapeutic agents or radiation or combinations thereof. Synergy may be shown, for example, by greater inhibition of tumor growth with combined treatment than would be expected from a treatment of only a BF₃ compound or the additive effect of treatment with a BF₃ compound and a chemotherapeutic agent or radiation. Preferably, synergy is demonstrated by remission of the cancer where remission is not expected from treatment either from a single agent BF₃ compound or with treatment using an additive combination of a BF₃ compound and a chemotherapeutic agent or radiation.

The method for inhibiting growth of tumor cells using a BF₃ compound and a combination of chemotherapy or radiation or both comprises administering the BF₃ compound before, during, or after commencing chemotherapy or radiation therapy, as well as any combination thereof (i.e., before and during, before and after, during and after, or before, during, and after commencing the chemotherapy and/or radiation therapy). For example, the BF₃ compound is typically administered between 1 and 60 days, preferably between 3 and 40 days, more preferably between 5 and 12 days before commencing radiation therapy and/or chemotherapy. However, depending on the treatment protocol and the specific patient's needs, the method is performed in a manner that will provide the most efficacious treatment and ultimately prolong the life of the patient.

The administration of chemotherapeutic agents can be accomplished in a variety of ways including systemically by the parenteral and enteral routes. In one embodiment, the BF₃ compound(s) and the chemotherapeutic agent are administered as separate molecules. Particular examples of chemotherapeutic agents or chemotherapy include cisplatin, dacarbazine (DTIC), dactinomycin, mechlorethamine (nitrogen mustard), streptozocin, cyclophosphamide, carmustine (BCNU), lomustine (CCNU), doxorubicin (adriamycin), daunorubicin, procarbazine, mitomycin, cytarabine, etoposide, methotrexate, 5-fluorouracil, vinblastine, vincristine, bleomycin, paclitaxel (taxol), docetaxel (taxotere), aldesleukin, asparaginase, busulfan, carboplatin, cladribine, dacarbazine, floxuridine, fludarabine, hydroxyurea, ifosfamide, interferon alpha, leuprolide, megestrol, melphalan, mercaptopurine, plicamycin, mitotane, pegaspargase, pentostatin, pipobroman, plicamycin, streptozocin, tamoxifen, teniposide, testolactone, thioguanine, thiotepa, uracil mustard, vinorelbine, gemcitabine, chlorambucil, taxol and combinations thereof.

The source of radiation, used in combination with a BF₃ compound, can be either external or internal to the patient being treated. When the source is external to the patient, the therapy is known as external beam radiation therapy (EBRT). When the source of radiation is internal to the patient, the treatment is called brachytherapy (BT). Additionally, BNCT and Proton Boron Fusion Therapy are contemplated herein.

The above-described therapeutic regimens may be further combined with additional cancer treating agents and/or regimes, for example additional chemotherapy, cancer vaccines, signal transduction inhibitors, agents useful in treating abnormal cell growth or cancer, antibodies (e.g., Anti-CTLA-4 antibodies as described in WO/2005/092380 (Pfizer)) or other ligands that inhibit tumor growth by binding to GF-1N, and cytokines.

When the mammal is subjected to additional chemotherapy, chemotherapeutic agents described above may be used. Additionally, growth factor inhibitors, biological response modifiers, anti-hormonal therapy, selective estrogen receptor modulators (SERMs), angiogenesis inhibitors, and anti-androgens may be used. For example, anti-hormones, for example anti-estrogens such as Nolvadex (tamoxifen) or, anti-androgens such as Casodex (4′-cyano-3-(4-fluorophenylsulphonyl)-2-hydroxy-2-methyl-3-(trifluoromethyl)propionanilide) may be used.

The above therapeutic approaches can be combined with any one of a wide variety of surgical, chemotherapy or radiation therapy regimens. The therapeutic approaches of the invention can enable the use of reduced dosages of chemotherapy (or other therapies) and/or less frequent administration, an advantage for all patients and particularly for those that do not tolerate the toxicity of the chemotherapeutic agent well.

### Exemplary Embodiments

1) A composition comprising a chemical structure as follows:

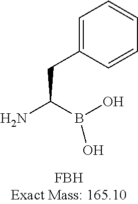 [SMILES: N[C@@H](Cc1ccccc1)B(O)O]

2) A composition comprising a chemical structure as follows:

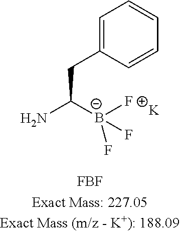 [SMILES: N[C@@H](Cc1ccccc1)[B-](F)(F)F ; [K+]]

3) A composition comprising a chemical structure as follows:

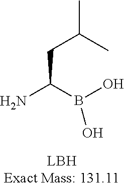

4) A composition comprising a chemical structure as follows:

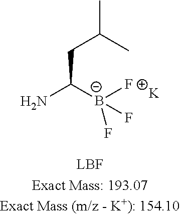

5) A composition comprising a chemical structure as follows:

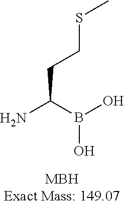

6) A composition comprising a chemical structure as follows:

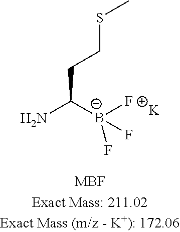

7) A composition comprising a chemical structure as follows:

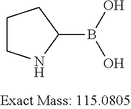

8) A composition comprising a chemical structure as follows:

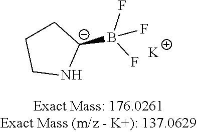

9) A composition comprising a chemical structure as follows:

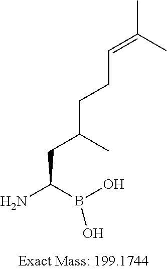

10) A composition comprising a chemical structure as follows:

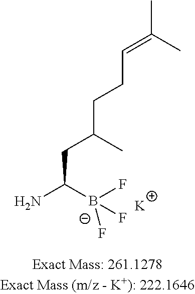

11) A kit comprising the composition of claim 2.
12) A kit comprising the composition of claim 3.
13) A kit comprising the composition of claim 4.
14) A kit comprising the composition of claim 5.
15) A kit comprising the composition of claim 6.
16) A kit comprising the composition of claim 7.
17) A kit comprising the composition of claim 8.
18) A kit comprising the composition of claim 9.
19) A kit comprising the composition of claim 10.
20) A method of producing a composition of claim 1.
21) A method of producing a composition of claim 2.
22) A method of producing a composition of claim 3.
23) A method of producing a composition of claim 4.
24) A method of producing a composition of claim 5.
25) A method of producing a composition of claim 6.
26) A method of producing a composition of claim 7.
27) A method of producing a composition of claim 8.
28) A method of producing a composition of claim 9.
29) A method of producing a composition of claim 10.
30) A Dosage Unit form comprising a composition of claim 1.
31) A Dosage Unit form comprising a composition of claim 2.
32) A Dosage Unit form comprising a composition of claim 3.
33) A Dosage Unit form comprising a composition of claim 4.
34) A Dosage Unit form comprising a composition of claim 5.
35) A Dosage Unit form comprising a composition of claim 6.
36) A Dosage Unit form comprising a composition of claim 7.
37) A Dosage Unit form comprising a composition of claim 8.
38) A Dosage Unit form comprising a composition of claim 9.
39) A Dosage Unit form comprising a composition of claim 10.
40) A method of performing Neutron Capture Therapy in the treatment of human cancer comprising:
   a. synthesizing a Human Unit Dose of a BF₃ Compound;
   b. injecting the BF₃ Compound into a tumor, whereby the BF₃ Compound accumulates into a cell; and
   c. irradiating the BF₃ Compound with neutrons.
41) The method of claim 40, wherein the composition is selected from the group consisting of the compositions in claim(s) 1, 2, 3, 4, 5, 6, 7, 8, 9, and 10.
42) The method of claim 40, wherein the Neutron Capture Therapy is Boron Neutron Capture Therapy.
43) The method of claim 40, wherein the irradiation comprises epithermal neutrons.
44) A method of performing Proton Boron Fusion Therapy in the treatment of human cancer comprising:
   a. synthesizing a Human Unit Dose of a BF₃ Compound;
   b. injecting the BF₃ Compound into a tumor, whereby the BF₃ Compound accumulates into a cell; and
   c. irradiating the BF₃ Compound with protons.
45) The method of claim 44, wherein the composition is selected from the group consisting of the compositions in claim(s) 1, 2, 3, 4, 5, 6, 7, 8, 9, and 10.
46) A method of synthesizing a BF₃ Compound by a process comprising,

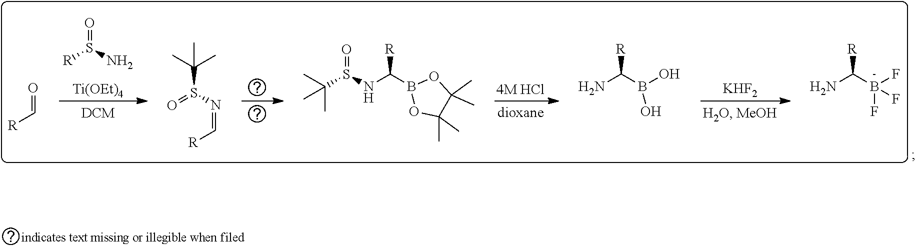

wherein R=

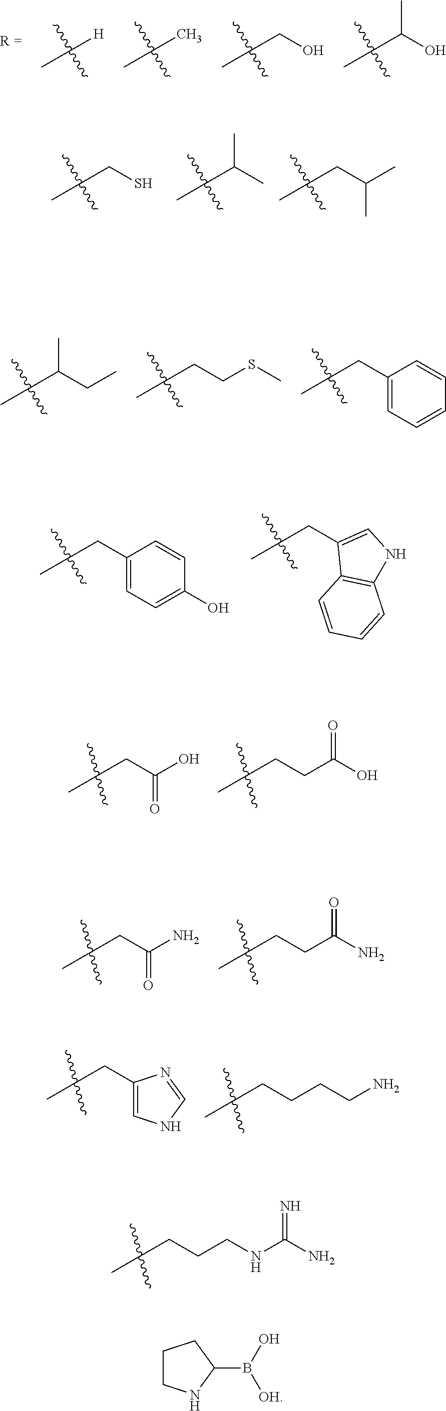

47) A method of synthesizing a BF₃ Compound comprising L-Leucine mimetics by a process comprising,

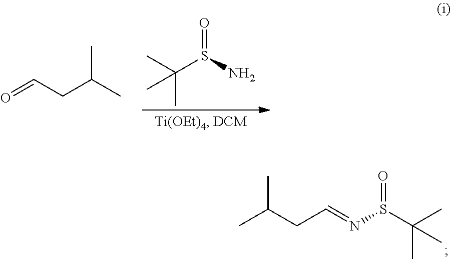

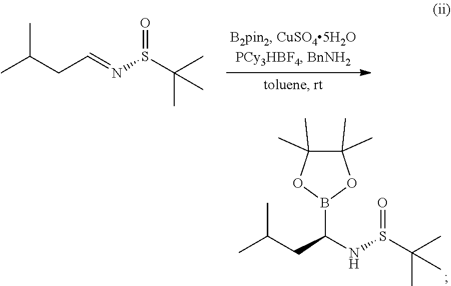 [SMILES: CC(C)C/C=N/[S@](=O)C(C)(C)C ; CC(C)C[C@H](N[S@](=O)C(C)(C)C)B1OC(C)(C)C(C)(C)O1]

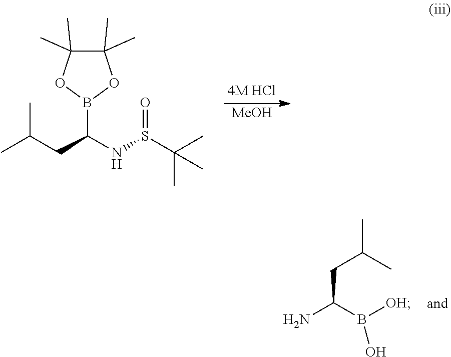

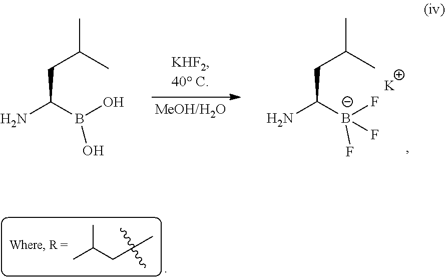

48) A method of synthesizing a BF₃ Compound comprising L-Methionine mimetics by a process comprising,

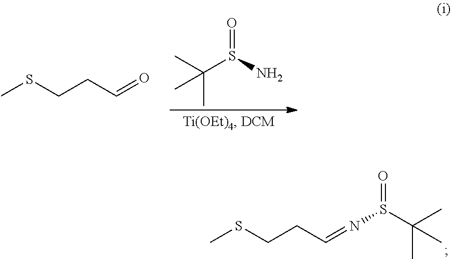

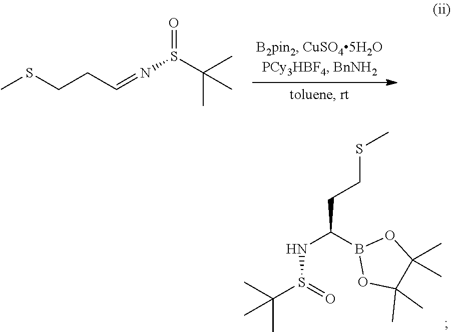 [SMILES: CSCCC=N[S@](=O)C(C)(C)C ; CSCC[C@H](N[S@](=O)C(C)(C)C)B1OC(C)(C)C(C)(C)O1]

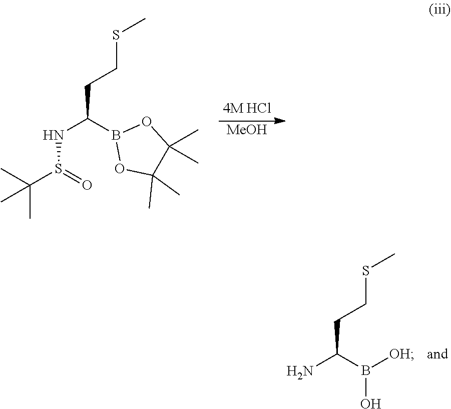

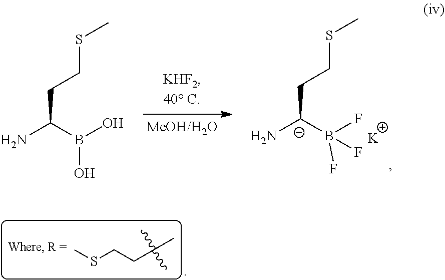

49) A method of synthesizing a BF₃ Compound comprising L-Proline mimetics by a process comprising,

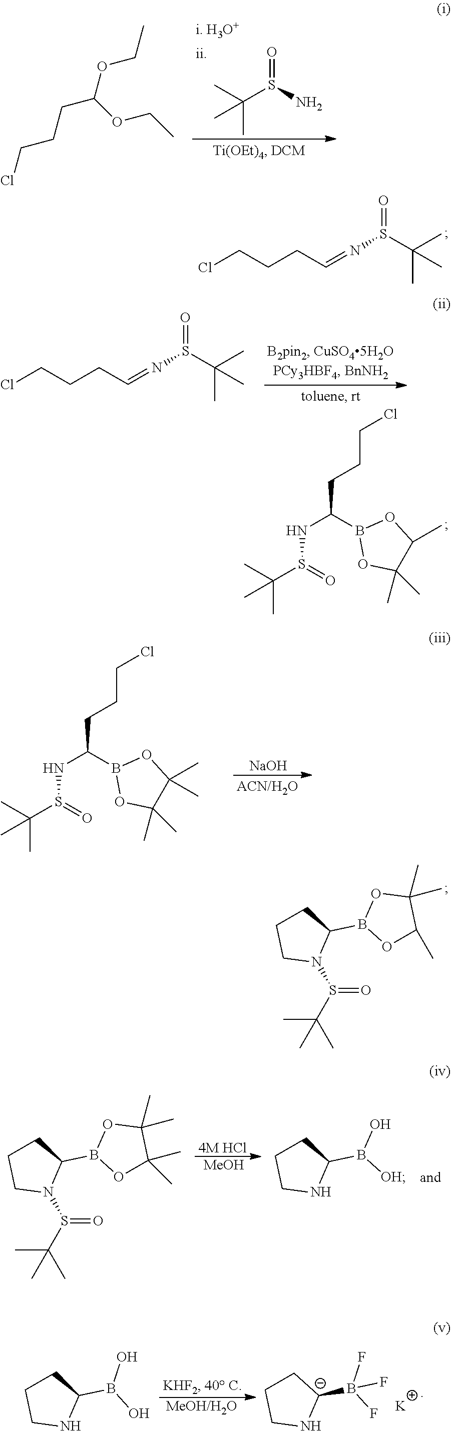

50) A method of synthesizing a BF₃ Compound comprising Citronells derivatives by a process comprising,

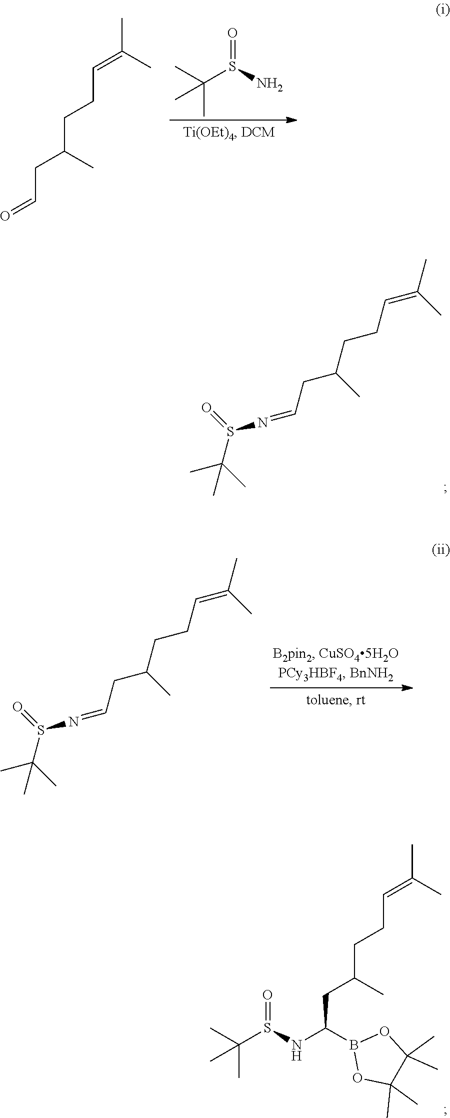

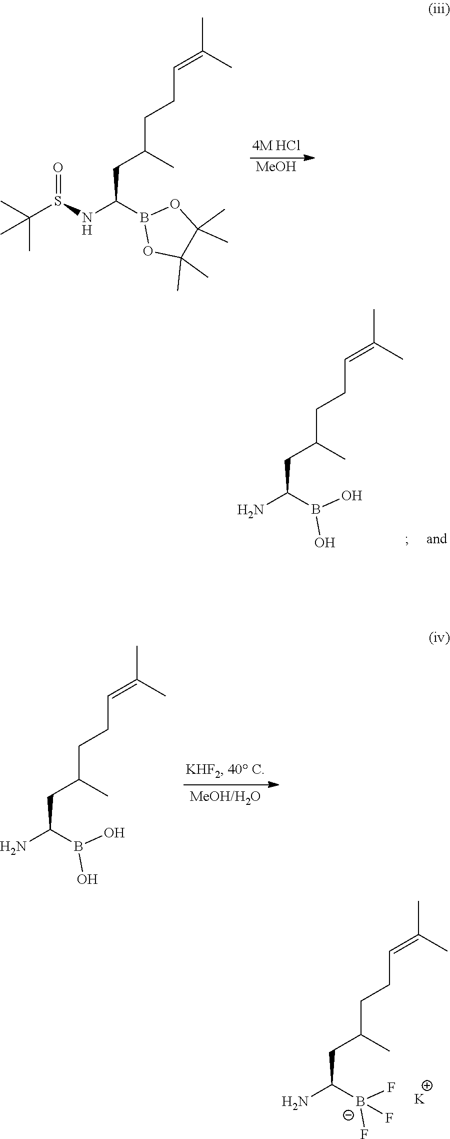 [SMILES: "MeOH" ; CC(C)=CCCC(C)C[C@H](N[S@](=O)C(C)(C)C)B1OC(C)(C)C(C)(C)O1 ; CC(C)=CCCC(C)C[C@H](N)B(O)O ; CC(C)=CCCC(C)C[C@H](N)B(O)O ; CC(C)=CCCC(C)C[C@H](N)[B-](F)(F)F ; [K+]]

51) A method of synthesizing a BF₃ Compound comprising a member of the Bortezomib family by a process comprising,

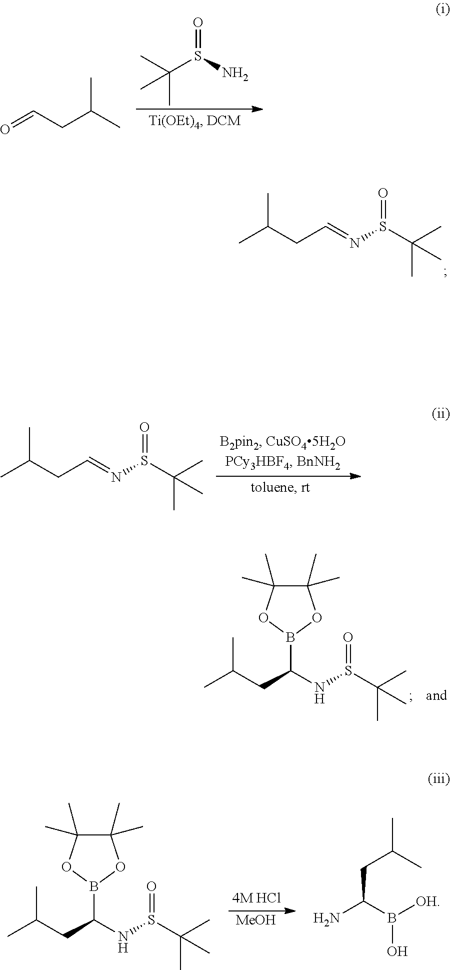

52) The method of claim 51, further comprising the synthesis of a compound having the following chemical structure:

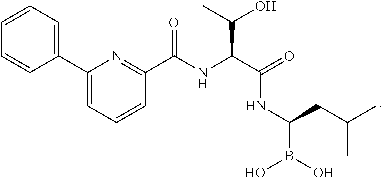 [SMILES: CC(C)C[C@H](NC(=O)[C@@H](NC(=O)c1cccc(-c2ccccc2)n1)C(C)O)B(O)O]

53) The method of claim 51, further comprising the synthesis of a compound having the following chemical structure:

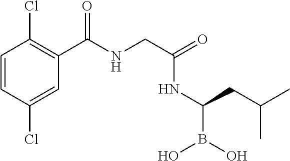 [SMILES: CC(C)C[C@H](NC(=O)CNC(=O)c1cc(Cl)ccc1Cl)B(O)O]

54) The method of claim 51, further comprising the synthesis of a compound having the following chemical structure:

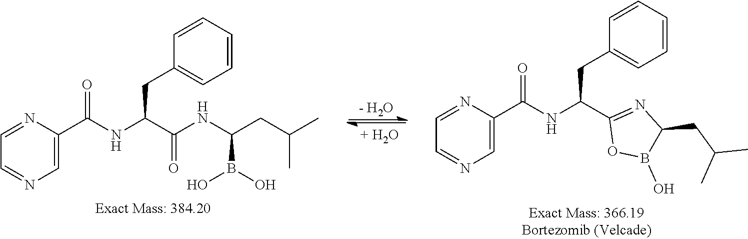

### EXAMPLES

Various aspects of the invention are further described and illustrated by way of the several examples that follow, none of which is intended to limit the scope of the invention.

### Example 1: Total Synthesis of FBH and FBF

FBH and FBF were synthesized in the following manner. Briefly, to a solution of 38 mL of methylene chloride (DCM) is added 2 g of phenylacetaldehyde and 7 mL of titanium ethoxide. After five (5) minutes of stirring, 2.02 g of (R)-(−)-tert-butylsulfinamide is added to the reaction. The reaction is refluxed at 40° C. for three (3) hours, then the temperature was brought down to 30° C. and allowed to stir overnight. After heating, the reaction is complete as observed by LCMS. Magnesium sulfate was added to the reaction and allowed to stir for five (5) minutes. The reaction mixture was then filtered through celite and concentrated to afford the target material:

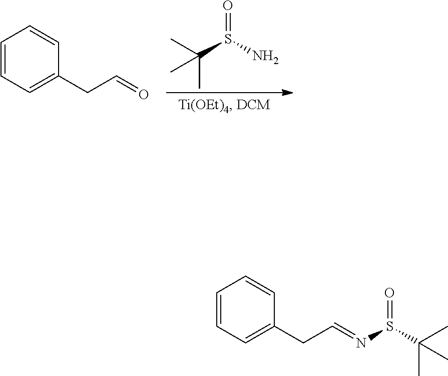

Then, to a solution of 5 mL of toluene at room temperature is added 143 mg of copper II sulfate pentahydrate (CuSO₄·5H₂O), 409 μL of benzylamine (BnNH₂), and 315 mg of tricyclohexylphosphonium tetrafluoroborate (Cy₃PHBF₄). After stirring for 15 minutes, an additional 45 mL of toluene was added followed by 5.6 g of (S)-2-methyl-N-[(1Z)-2-phenylethylidene]propane-2-sulfinamide and 7.6 g of bis(pinacolato)diboron. After stirring for twelve (12) hours, the reaction is complete as observed by LCMS. The reaction mixture is then diluted with 50 mL of ethyl acetate (EtOAc) and filtered through a silica plug. The solvent is removed under reduced pressure and the concentrate purified via flash column chromatography (FCC) to yield the target material.

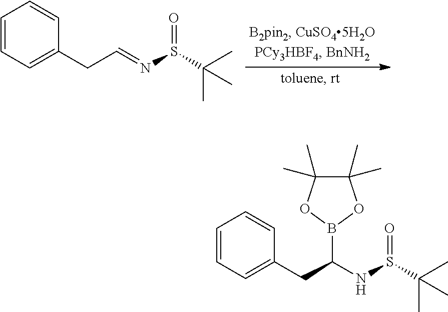

Then, to a solution containing 40 mL of methanol (MeOH) at −83° C. is added 4 g of 2-methyl-N-[(1R)-2-phenyl-1-(4,4,5,5-tetramethyl-1,3,2-dioxaborolan-2-yl)ethyl]propane-2-sulfonamide. Next, 6 mL of 4M hydrochloric acid (HCl) in 1,4-dioxane was slowly added to the reaction. After two (2) hours of stirring, the reaction is complete as observed by LCMS. The methanol was then removed under reduced pressure and the concentrate was diluted with saturated sodium bicarbonate and ethyl acetate. The aqueous layer was then separated and placed onto a preparative HPLC to afford the following target material:

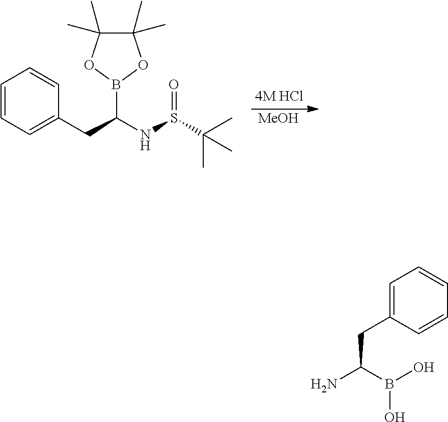

Finally, to a flask containing 300 mg of L-phenylalanine boronic acid (FBH) is added 6 mL of methanol (MeOH) and 303 μL of water. Then, 469 mg of potassium bifluoride (KHF₂) is added and the solution is heated to 40° C. After two (2) hours of heating, the reaction is complete as observed by LCMS. The methanol is then removed under reduced pressure and placed onto a preparative HPLC to afford the following target material [(1R)-1-amino-2-phenylethyl]trifluoroboranuide (FBF):

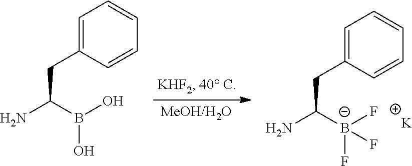 [SMILES: N[C@@H](Cc1ccccc1)B(O)O ; N[C@@H](Cc1ccccc1)[B-](F)(F)F ; [K+]]

The resulting composition denoted FBH is set forth in FIG. 2 and presented below:

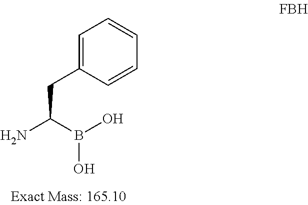 [SMILES: N[C@@H](Cc1ccccc1)B(O)O]

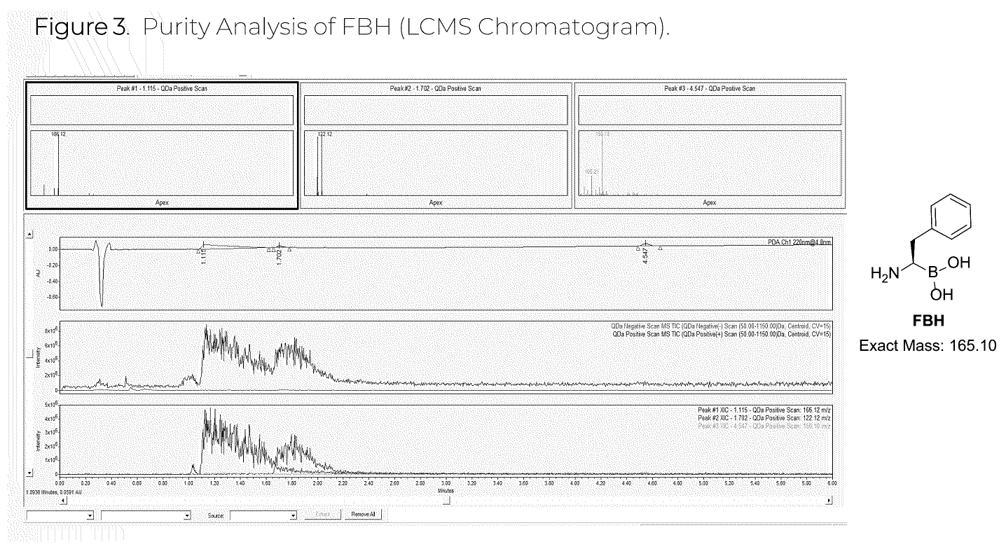

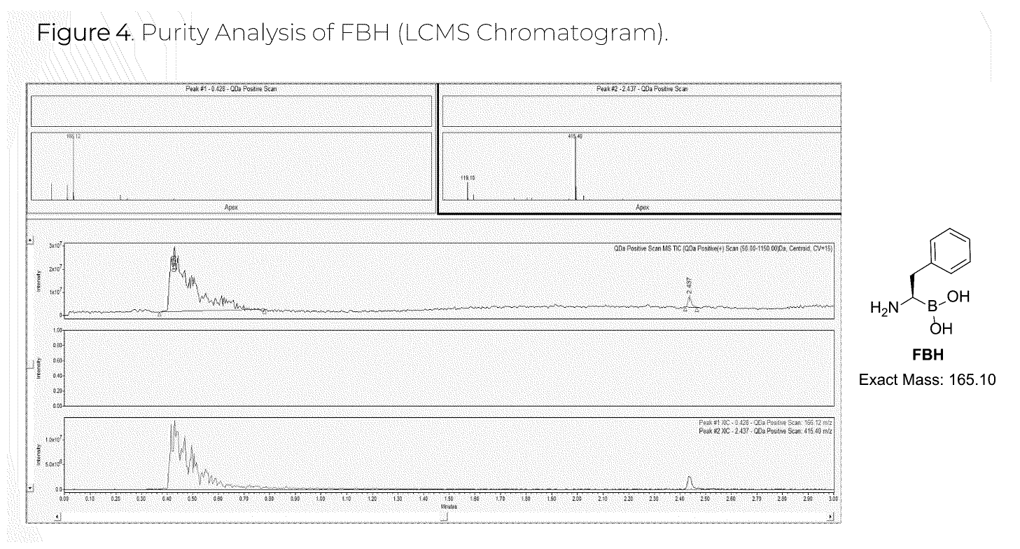

FBH was analyzed by LC/MS to confirm the molecular weight and the purity was determined after the preparation of aqueous solution. Briefly, an FBH was analyzed using Luna Omega Polar C18 column (2.1×150 mm, Phenomenex) maintained at 40° C. and with the flow rate of 0.5 ml/min. The bound test article was eluted in the gradient of acetonitrile—0.1% TFA. The resulting purity analysis of FBH is shown in FIG. 3 and FIG. 4.

The resulting composition denoted FBF is set forth in FIG. 2 and presented below:

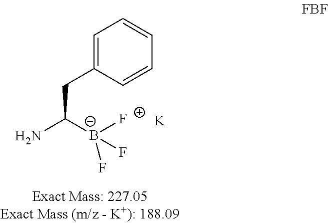

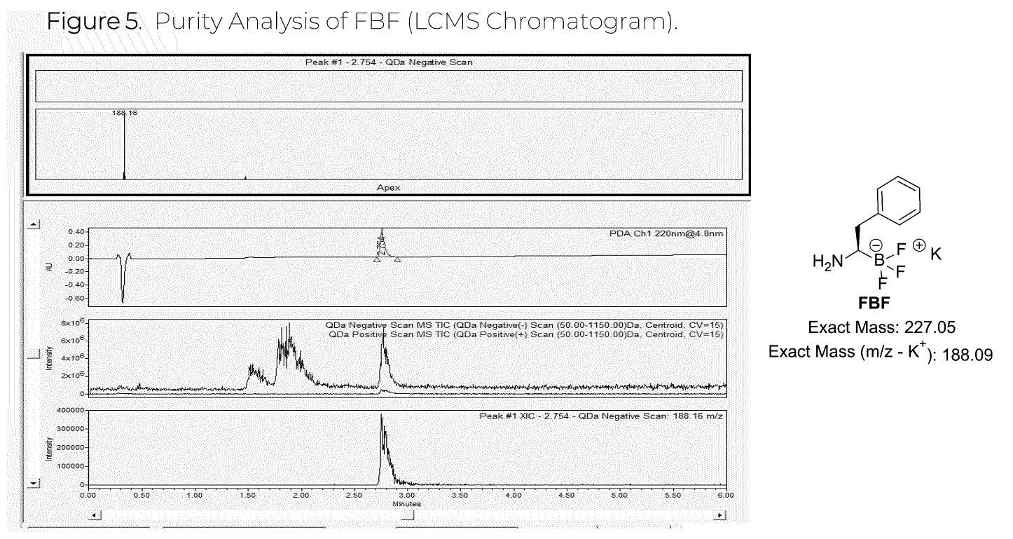

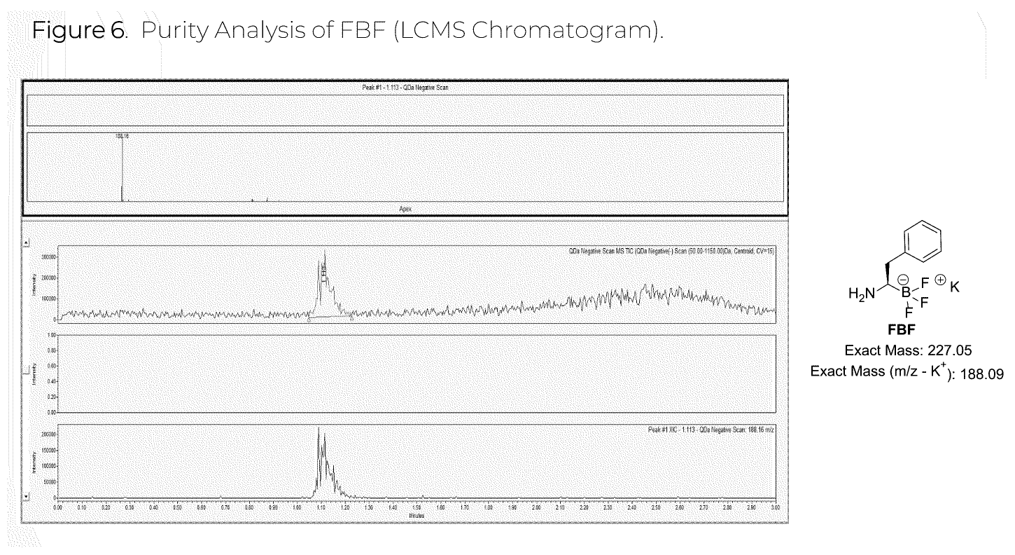

FBF was analyzed by LC/MS to confirm the molecular weight and the purity was determined after the preparation of aqueous solution. Briefly, a FBF was analyzed using Luna Omega Polar C18 column (2.1×150 mm, Phenomenex) maintained at 40° C. and with the flow rate of 0.5 ml/min. The bound test article was eluted in the gradient of acetonitrile—0.1% TFA. The resulting purity analysis of FBF is shown in FIG. 5 and FIG. 6.

### Example 2: Total Synthesis of LBH and LBF

LBH and LBF were synthesized in the following manner. Briefly, to a solution of 30 mL of methylene chloride (DCM) is added 2 g of isovaleraldehyde and 9.7 mL of titanium ethoxide. After five (5) minutes of stirring, 2.81 g of (R)-(−)-tert-butylsulfinamide is added to the reaction. The reaction is refluxed at 40° C. for three (3) hours, then the temperature was brought down to 30° C. and allowed to stir overnight. After heating, the reaction is complete as observed by LCMS. Magnesium sulfate was added to the reaction and allowed to stir for five (5) minutes. The reaction mixture was then filtered through celite and concentrated to afford the target material:

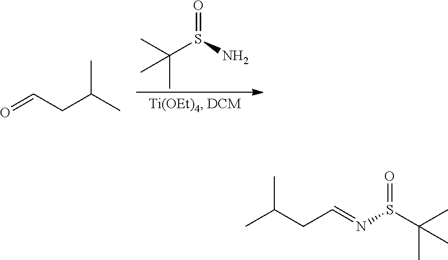

Then, to a solution of 5 mL of toluene at room temperature is added 70 mg of copper II sulfate pentahydrate (CuSO₄·5H₂O), 127 μL of benzylamine (BnNH₂), and 98 mg of tricyclohexylphosphonium tetrafluoroborate (Cy₃PHBF₄). After stirring for 15 minutes, an additional 40 mL of toluene was added followed by 4.4 g of (R,E)-2-methyl-N-(3-methylbutylidene)propane-2-sulfinamide and 11.8 g of bis(pinacolato)diboron. After stirring for twelve (12) hours, the reaction is complete as observed by LCMS. The reaction mixture is then diluted with 50 mL of ethyl acetate (EtOAc) and filtered through a silica plug. The solvent is removed under reduced pressure and the concentrate purified via flash column chromatography (FCC) to yield the target material:

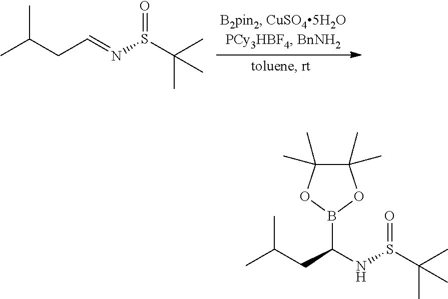 [SMILES: CC(C)C/C=N/[S@](=O)C(C)(C)C ; CC(C)C[C@H](N[S@](=O)C(C)(C)C)B1OC(C)(C)C(C)(C)O1]

Then, to a solution containing 60 mL of methanol (MeOH) at −83° C. is added 6 g of (R)-2-methyl-N-[(1R)-3-methyl-1-(4,4,5,5-tetramethyl-1,3,2-dioxaborolan-2-yl)butyl]propane-2-sulfinamide. Next, 9.5 mL of 4M hydrochloric acid (HCl) in 1,4-dioxane was slowly added to the reaction. After two (2) hours of stirring, the reaction is complete as observed by LCMS. The methanol was then removed under reduced pressure and the concentrate was diluted with saturated sodium bicarbonate and ethyl acetate. The aqueous layer was then separated and placed onto a preparative HPLC to afford the following target material:

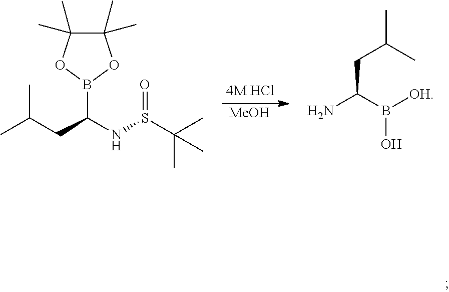

Finally, to a flask containing 150 mg of L-leucine boronic acid (LBH) is added 3 mL of methanol (MeOH) and 150 μL of water. Then, 295 mg of potassium bifluoride (KHF₂) is added and the solution is heated to 40° C. After two (2) hours of heating, the reaction is complete as observed by LCMS. The methanol is then removed under reduced pressure and placed onto a preparative HPLC to afford the following target material potassium [(1R)-1-amino-3-methylbutyl]trifluoroboranuide (LBF):

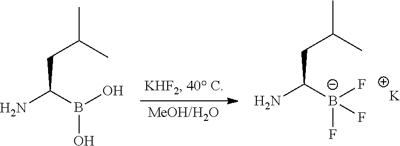

The resulting synthesis and composition denoted LBH is shown in FIG. 7 and is set forth below:

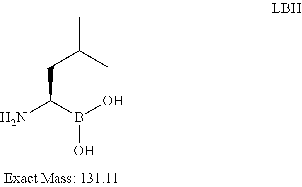

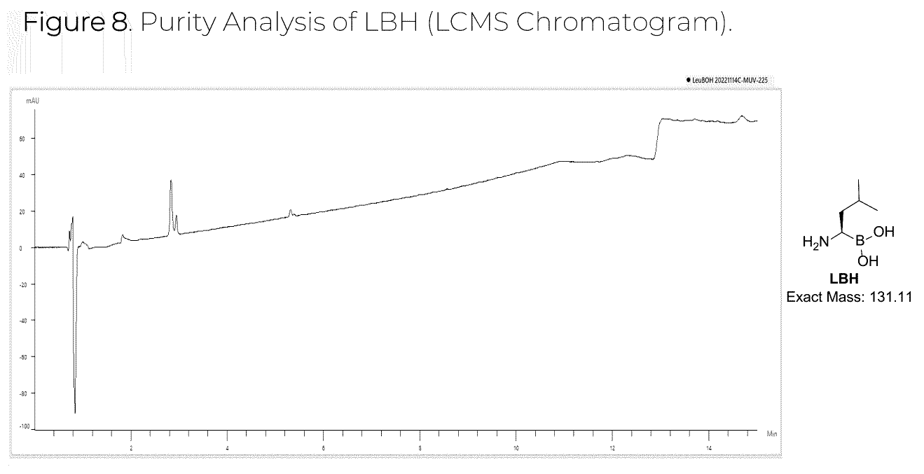

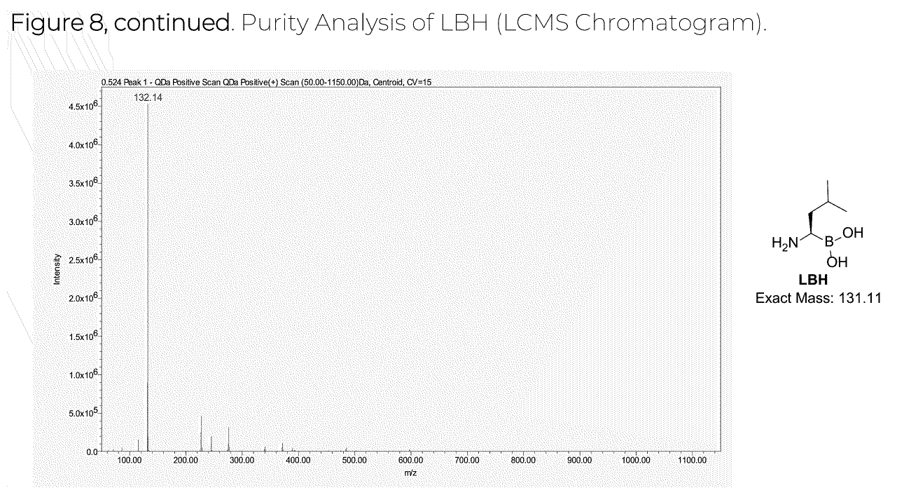

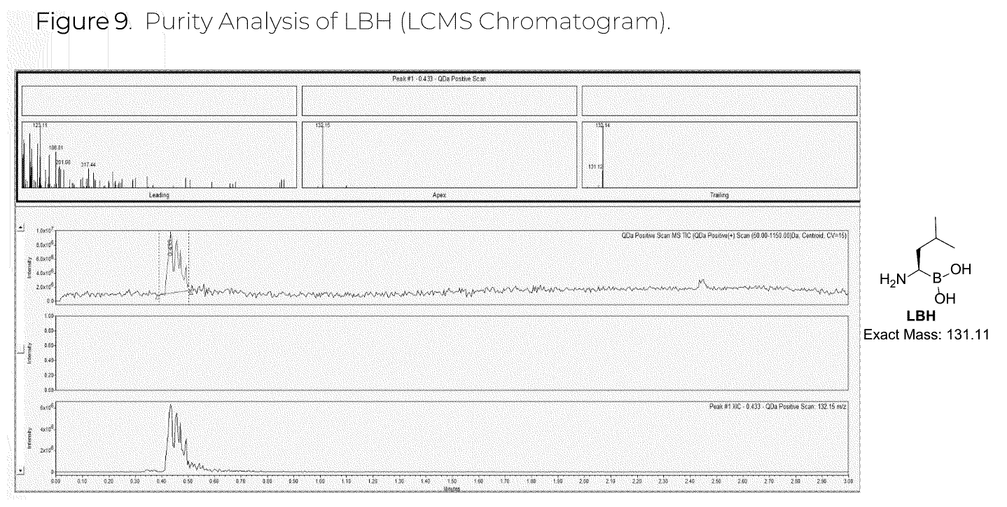

LBH was analyzed by LC/MS to confirm the molecular weight and the purity was determined after the preparation of aqueous solution. Briefly, LBH was analyzed using Luna Omega Polar C18 column (2.1×150 mm, Phenomenex) maintained at 40° C. and with the flow rate of 0.5 ml/min. The bound test article was eluted in the gradient of acetonitrile—0.1% TFA. The resulting purity analysis of LBH is shown in FIG. 8 and FIG. 9.

The resulting synthesis and composition denoted LBF is shown in FIG. 7 and is set forth below:

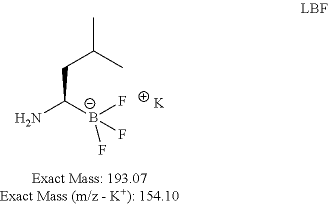

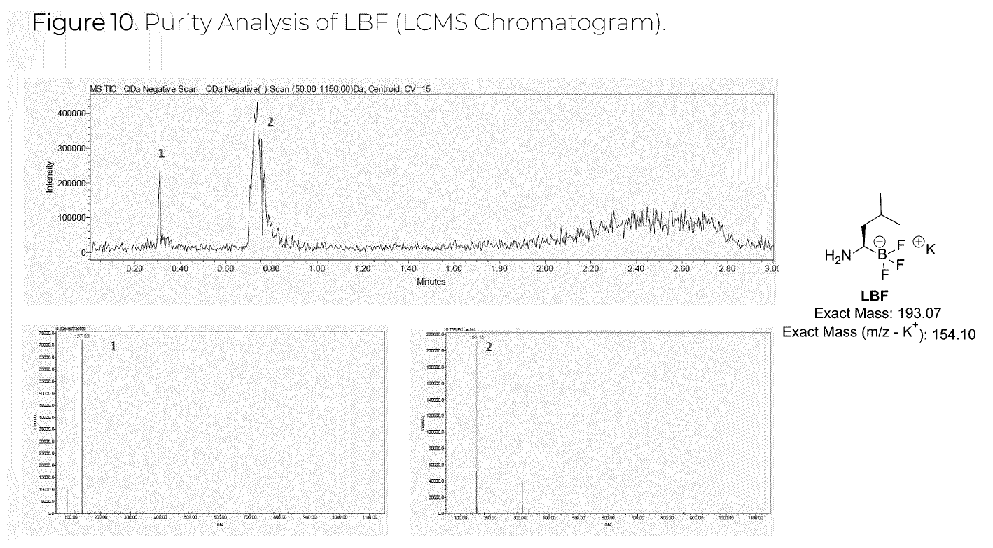

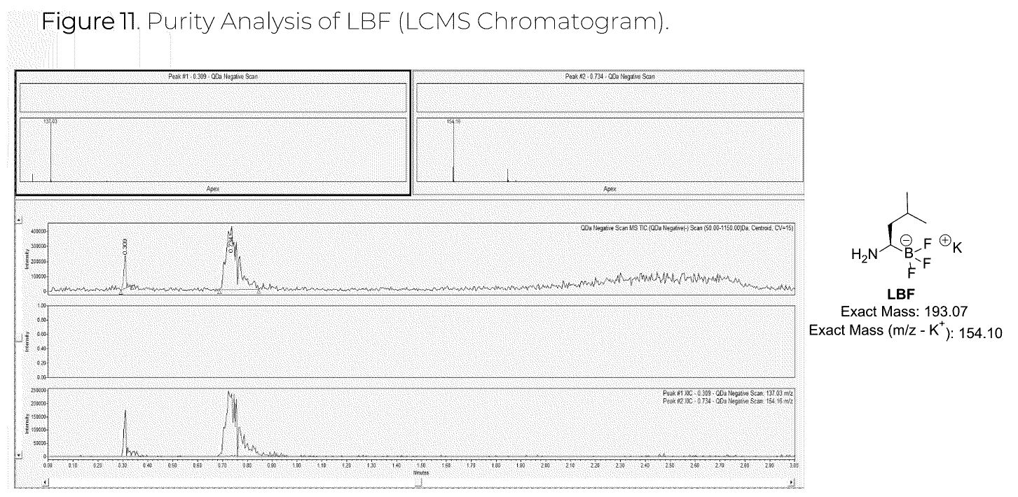

LBF was analyzed by LC/MS to confirm the molecular weight and the purity was determined after the preparation of aqueous solution. Briefly, LBF was analyzed using Luna Omega Polar C18 column (2.1×150 mm, Phenomenex) maintained at 40° C. and with the flow rate of 0.5 ml/min. The bound test article was eluted in the gradient of acetonitrile—0.1% TFA. The resulting purity analysis of LBF is shown in FIG. 10 and FIG. 11.

### Example 3: Total Synthesis of MBH and MBF

MBH and MBF were synthesized in the following manner. Briefly, to a solution of 45 mL of methylene chloride (DCM) is added 3 g of methional and 9 mL of titanium ethoxide. After five (5) minutes of stirring, 3.84 g of (R)-(−)-tert-butylsulfinamide is added to the reaction. The reaction is refluxed at 40° C. for three (3) hours, then the temperature was brought down to 30° C. and allowed to stir overnight. After heating, the reaction is complete as observed by LCMS. Magnesium sulfate was added to the reaction and allowed to stir for five (5) minutes. The reaction mixture was then filtered through celite and concentrated to afford the target material:

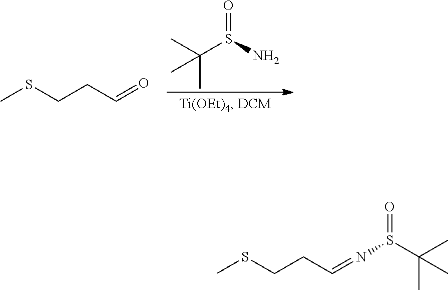

Then, to a solution of 5 mL of toluene at room temperature is added 518 mg of copper II sulfate pentahydrate (CuSO₄·5H₂O), 692 μL of benzylamine (BnNH₂), and 764 mg of tricyclohexylphosphonium tetrafluoroborate (Cy₃PHBF₄). After stirring for 15 minutes, an additional 55 mL of toluene was added followed by 5.9 g of (R)-2-methyl-N-(3-(methylthio)propylidene)propane-2-sulfinamide and 8.7 g of bis(pinacolato)diboron. After stirring for twelve (12) hours, the reaction is complete as observed by LCMS. The reaction mixture is then diluted with 50 mL of ethyl acetate (EtOAc) and filtered through a silica plug. The solvent is removed under reduced pressure and the concentrate purified via flash column chromatography (FCC) to yield the target material:

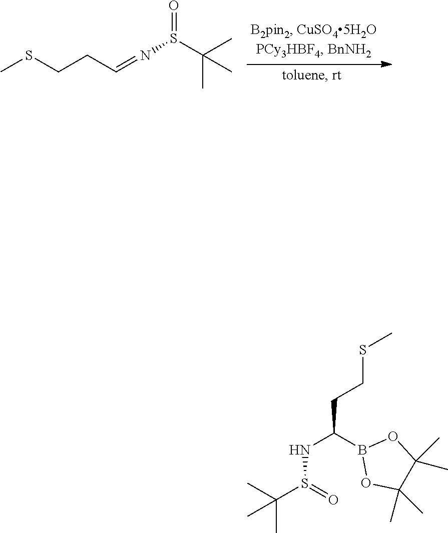 [SMILES: CSCCC=N[S@](=O)C(C)(C)C ; CSCC[C@H](N[S@](=O)C(C)(C)C)B1OC(C)(C)C(C)(C)O1]

Then, to a solution containing 73 mL of methanol (MeOH) at 0° C. is added 8 g of (R)-2-methyl-N—((R)-3-(methylthio)-1-(4,4,5,5-tetramethyl-1,3,2-dioxaborolan-2-yl)propyl)propane-2-sulfinamide. Next, 12.0 mL of 4M hydrochloric acid (HCl) in 1,4-dioxane was slowly added to the reaction. After two (2) hours of stirring, the reaction is complete as observed by LCMS. The methanol was then removed under reduced pressure and the concentrate was diluted with saturated sodium bicarbonate and ethyl acetate. The aqueous layer was then separated and placed onto a preparative HPLC to afford the following target material:

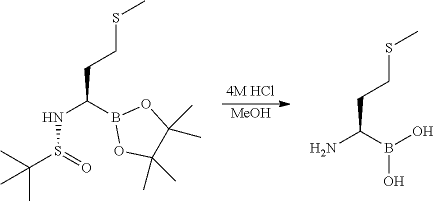

Finally, to a flask containing 150 mg of L-methionineboronic acid ((R)-(1-amino-3-(methylthio)propyl)boronic acid, MBH) is added 3 mL of methanol (MeOH) and 150 μL of water. Then, 259 mg of potassium bifluoride (KHF₂) is added and the solution is heated to 40° C. After two (2) hours of heating, the reaction is complete as observed by LCMS. The methanol is then removed under reduced pressure and placed onto a preparative HPLC to afford the following target material potassium (R)-3-(methylthio)-1-(trifluoro-λ⁴-boraneyl)propan-1-amine (MBF):

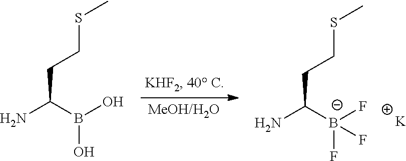

The resulting synthesis and composition denoted MBH is shown in FIG. 12 and is set forth below:

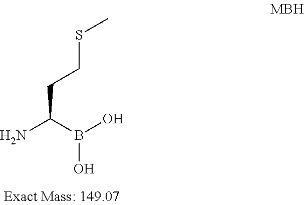

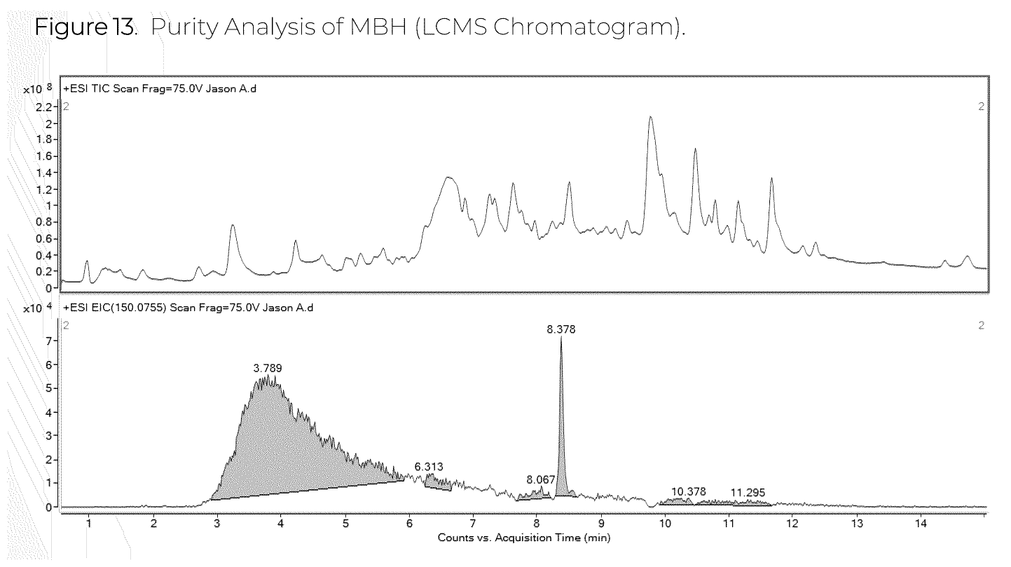

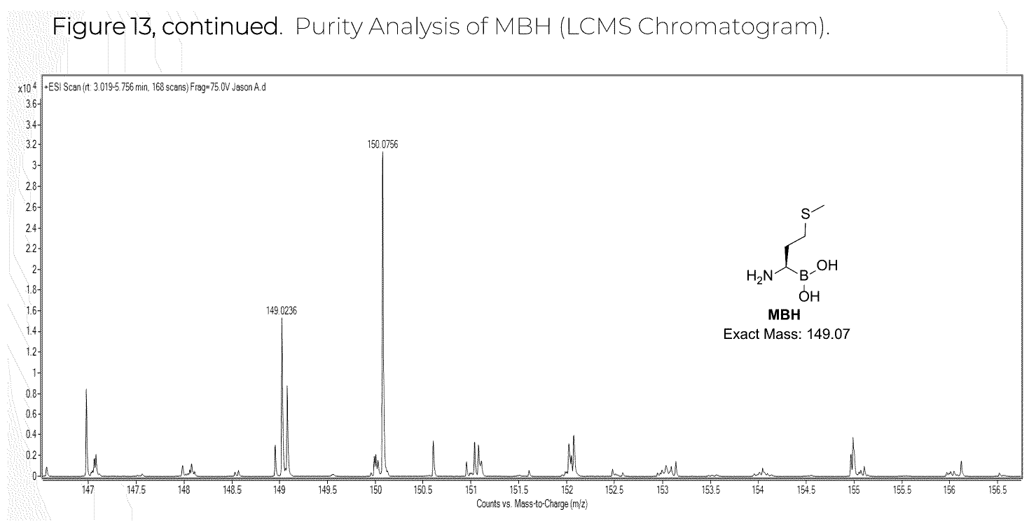

MBH is analyzed by LC/MS to confirm the molecular weight and the purity was determined after the preparation of aqueous solution. Briefly, MBH is analyzed using Luna Omega Polar C18 column (2.1×150 mm, Phenomenex) maintained at 40° C. and with the flow rate of 0.5 ml/min. The bound test article was eluted in the gradient of acetonitrile—0.1% TFA. The resulting purity analysis of MBH is shown in FIG. 13.

The resulting synthesis and composition denoted MBF is shown in FIG. 12 and is set forth below:

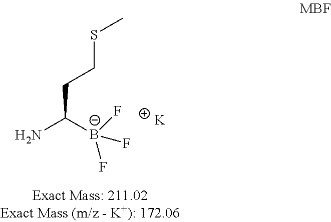

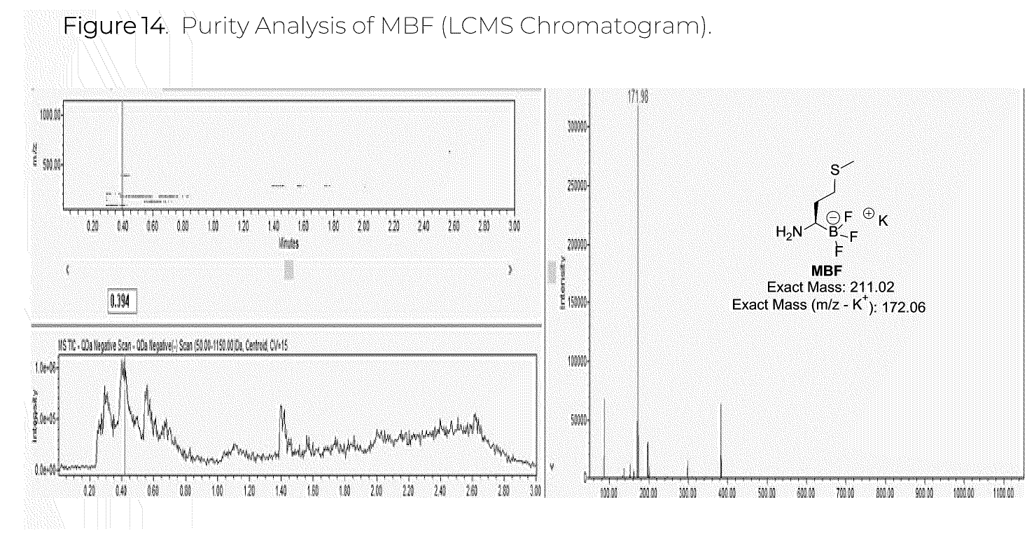

MBF is analyzed by LC/MS to confirm the molecular weight and the purity was determined after the preparation of aqueous solution. Briefly, MBF is analyzed using Luna Omega Polar C18 column (2.1×150 mm, Phenomenex) maintained at 40° C. and with the flow rate of 0.5 ml/min. The bound test article was eluted in the gradient of acetonitrile—0.1% TFA. The resulting purity analysis of MBF is shown in FIG. 14.

### Example 4: Total Synthesis of PBH and PBF

PBH and PBF were synthesized in the following manner. Briefly, to a solution of 47.5 mL of water is added 0.95 mL of hydrochloric acid and 4.6 g of 4-chloro-1,1-diethoxybutane. After stirring for two (2) hours, the solution was washed three (3) times with 20 mL of methylene chloride (DCM). The organic layers were combined and 6.4 mL of titanium ethoxide was added. After five (5) minutes of stirring, 4.3 g of (R)-(−)-tert-butylsulfinamide is added to the reaction. The reaction is refluxed at 40° C. for three (3) hours, then the temperature was brought down to 30° C. and allowed to stir overnight. After heating, the reaction is complete as observed by LCMS. Magnesium sulfate was added to the reaction and allowed to stir for five (5) minutes. The reaction mixture was then filtered through celite and concentrated to afford the target material:

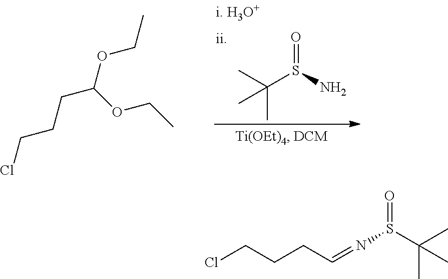

Subsequently, to a solution of 5 mL of toluene at room temperature is added 458 mg of copper II sulfate pentahydrate (CuSO₄·5H₂O), 556 μL of benzylamine (BnNH₂), and 675 mg of tricyclohexylphosphonium tetrafluoroborate (Cy₃PHBF₄). After stirring for 15 minutes, an additional 45 mL of toluene was added followed by 5.3 g of (R)—N-(4-chlorobutylidene)-2-methylpropane-2-sulfinamide and 7.8 g of bis(pinacolato)diboron. After stirring for twelve (12) hours, the reaction is complete as observed by LCMS. The reaction mixture is then diluted with 50 mL of ethyl acetate (EtOAc) and filtered through a silica plug. The solvent is removed under reduced pressure and the concentrate purified via flash column chromatography (FCC) to yield the target material:

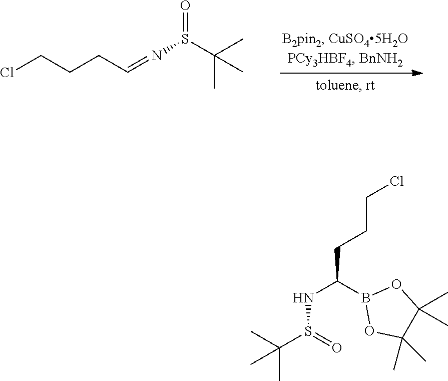 [SMILES: CC(C)(C)[S@@](=O)N=CCCCCl ; CC(C)(C)[S@@](=O)N[C@@H](CCCCl)B1OC(C)(C)C(C)(C)O1]

Then to a solution of 84 mL of tetrahydrofuran (THF) is added 8.5 g of (R)—N—((R)-4-chloro-1-(4,4,5,5-tetramethyl-1,3,2-dioxaborolan-2-yl)butyl)-2-methylpropane-2-sulfinamide and 2.1 g of potassium hydroxide and allowed to stir overnight. After stirring overnight, the reaction is complete as observed by LCMS. The reaction mixture was then filtered and concentrated to afford the target material:

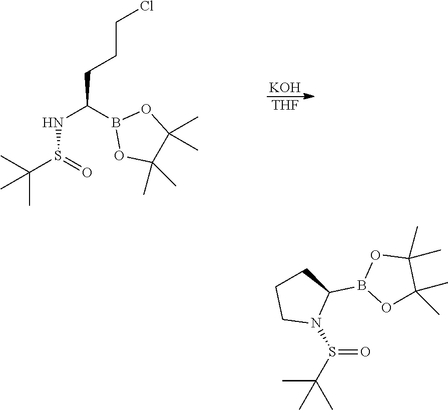 [SMILES: CC(C)(C)[S@@](=O)N[C@@H](CCCCl)B1OC(C)(C)C(C)(C)O1 ; "KOH" ; CC(C)(C)[S@@](=O)N1CCC[C@H]1B1OC(C)(C)C(C)(C)O1 ; "THF"]

Then, to a solution containing 75 mL of methanol (MeOH) at 0° C. is added 7.5 g of (R)-1-((R)-tert-butylsulfinyl)-2-(4,4,5,5-tetramethyl-1,3,2-dioxaborolan-2-yl)pyrrolidine. Next, 12.5 mL of 4M hydrochloric acid (HCl) in 1,4-dioxane was slowly added to the reaction. After two (2) hours of stirring, the reaction is complete as observed by LCMS. The methanol was then removed under reduced pressure and the concentrate was diluted with saturated sodium bicarbonate and ethyl acetate. The aqueous layer was then separated and placed onto a preparative HPLC to afford the following target material:

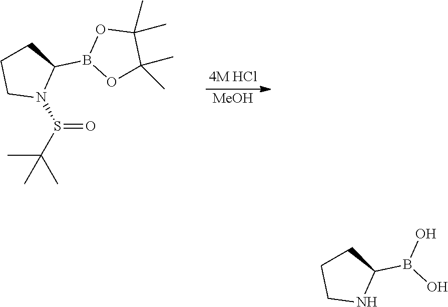 [SMILES: "MeOH" ; CC(C)(C)[S@@](=O)N1CCC[C@H]1B1OC(C)(C)C(C)(C)O1 ; OB(O)[C@@H]1CCCN1]

Finally, to a flask containing 150 mg of L-proline boronic acid ((R)-pyrrolidin-2-ylboronic acid, PBH) is added 3 mL of methanol (MeOH) and 150 μL of water. Then, 336 mg of potassium bifluoride (KHF₂) is added and the solution is heated to 40° C. After two (2) hours of heating, the reaction is complete as observed by LCMS. The methanol is then removed under reduced pressure and placed onto a preparative HPLC to afford the following target material potassium (R)-2-(trifluoro-λ⁴-boraneyl)pyrrolidine (PBF):

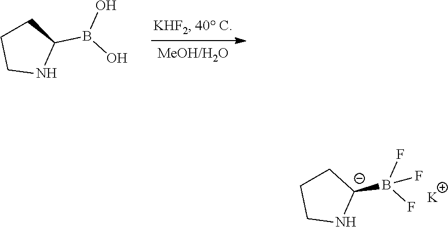

The resulting synthesis and composition(s) denoted PBH and PBF are shown in FIG. 15.

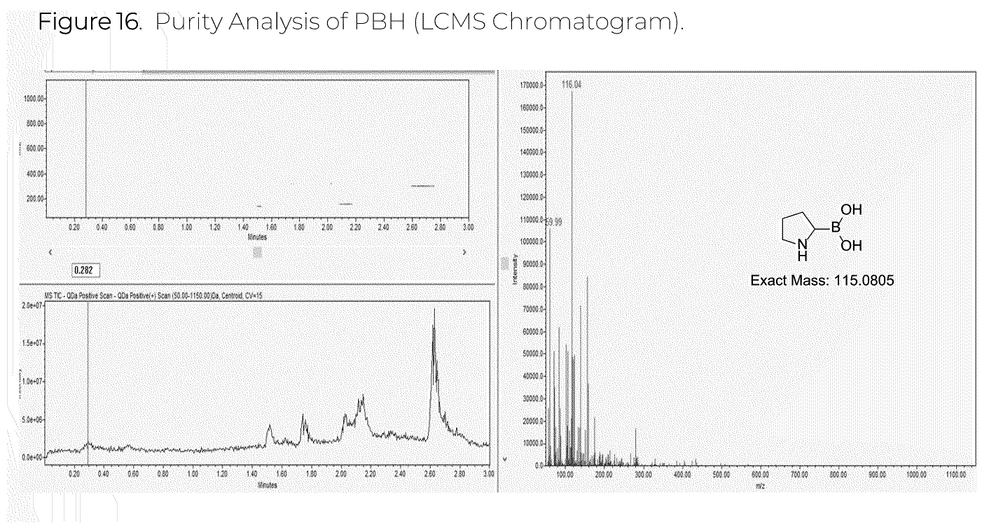

PBH is analyzed by LC/MS to confirm the molecular weight and the purity was determined after the preparation of aqueous solution. Briefly, PBH is analyzed using Luna Omega Polar C18 column (2.1×150 mm, Phenomenex) maintained at 40° C. and with the flow rate of 0.5 ml/min. The bound test article was eluted in the gradient of acetonitrile—0.1% TFA. The resulting purity analysis of PBH is shown in FIG. 16.

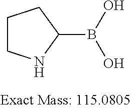

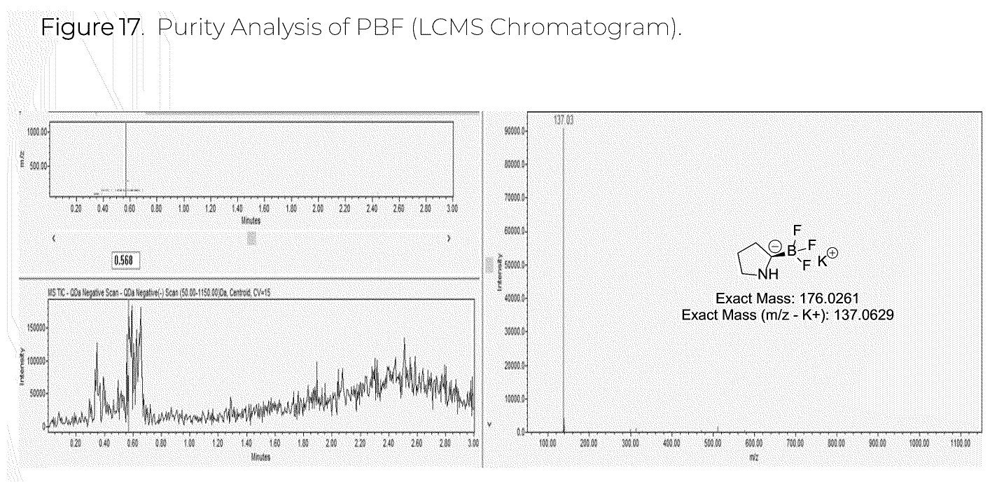

The resulting synthesis and composition denoted PBF is shown in FIG. 17 and is set forth below:

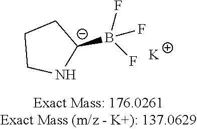

### Example 5: Total Synthesis of ((1R)-1-amino-3,7-dimethyloct-6-en-1-yl)boronic acid & Potassium ((1R)-1-amino-3,7-dimethyloct-6-en-1-yl)trifluoroborate (Citronella Derivatives)

((1R)-1-amino-3,7-dimethyloct-6-en-1-yl)boronic acid & potassium ((1R)-1-amino-3,7-dimethyloct-6-en-1-yl)trifluoroborate were synthesized in the following manner. Briefly, to a solution of 30 mL of methylene chloride (DCM) is added 3 g of citronellal and 4.9 mL of titanium ethoxide. After five (5) minutes of stirring, 2.8 g of (R)-(−)-tert-butylsulfinamide is added to the reaction. The reaction is refluxed at 40° C. for three (3) hours, then the temperature was brought down to 30° C. and allowed to stir overnight. After heating, the reaction is complete as observed by LCMS. Magnesium sulfate was added to the reaction and allowed to stir for five (5) minutes. The reaction mixture was then filtered through celite and concentrated to afford the target material:

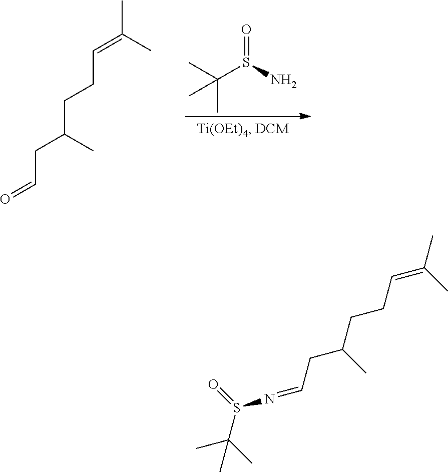

Subsequently, to a solution of 5 mL of toluene at room temperature is added 350 mg of copper II sulfate pentahydrate (CuSO₄·5H₂O), 425 μL of benzylamine (BnNH₂), and 516 mg of tricyclohexylphosphonium tetrafluoroborate (Cy₃PHBF₄). After stirring for 15 minutes, an additional 35 mL of toluene was added followed by 5 g of (R)—N-(3,7-dimethyloct-6-en-1-ylidene)-2-methylpropane-2-sulfinamide and 5.9 g of bis(pinacolato)diboron. After stirring for twelve (12) hours, the reaction is complete as observed by LCMS. The reaction mixture is then diluted with 50 mL of ethyl acetate (EtOAc) and filtered through a silica plug. The solvent is removed under reduced pressure and the concentrate purified via flash column chromatography (FCC) to yield the target material:

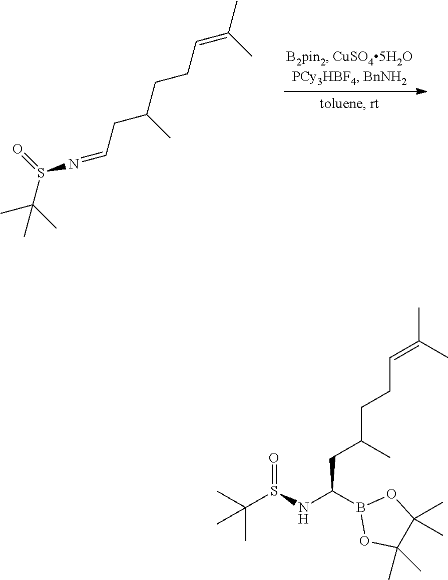 [SMILES: CC(C)=CCCC(C)CC=N[S@](=O)C(C)(C)C ; CC(C)=CCCC(C)C[C@H](N[S@](=O)C(C)(C)C)B1OC(C)(C)C(C)(C)O1]

Then, to a solution containing 37 mL of methanol (MeOH) at 0° C. is added 3.7 g of (R)—N-((1R)-3,7-dimethyl-1-(4,4,5,5-tetramethyl-1,3,2-dioxaborolan-2-yl)oct-6-en-1-yl)-2-methylpropane-2-sulfinamide. Next, 5.0 mL of 4M hydrochloric acid (HCl) in 1,4-dioxane was slowly added to the reaction. After two (2) hours of stirring, the reaction is complete as observed by LCMS. The methanol was then removed under reduced pressure and the concentrate was diluted with saturated sodium bicarbonate and ethyl acetate. The aqueous layer was then separated and placed onto a preparative HPLC to afford the following target material:

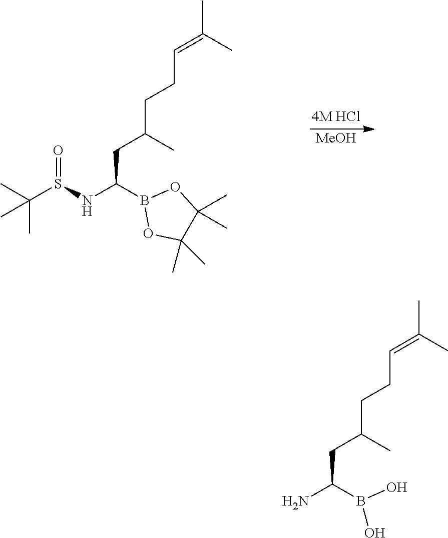 [SMILES: "MeOH" ; CC(C)=CCCC(C)C[C@H](N[S@](=O)C(C)(C)C)B1OC(C)(C)C(C)(C)O1 ; CC(C)=CCCC(C)C[C@H](N)B(O)O]

Finally, to a flask containing 1.0 g of ((1R)-1-amino-3,7-dimethyloct-6-en-1-yl)boronic acid is added 20 mL of methanol (MeOH) and 1 mL of water. Then, 1.3 g of potassium bifluoride (KHF₂) is added and the solution is heated to 40° C. After two (2) hours of heating, the reaction is complete as observed by LCMS. The methanol is then removed under reduced pressure and placed onto a preparative HPLC to afford the following target material potassium ((1R)-1-amino-3,7-dimethyloct-6-en-1-yl)trifluoroborate:

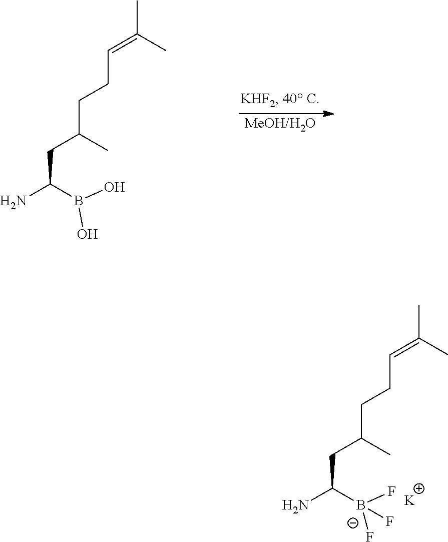

The resulting synthesis and composition(s) denoted ((1R)-1-amino-3,7-dimethyloct-6-en-1-yl)boronic acid and Potassium ((1R)-1-amino-3,7-dimethyloct-6-en-1-yl)trifluoroborate are shown in FIG. 18.

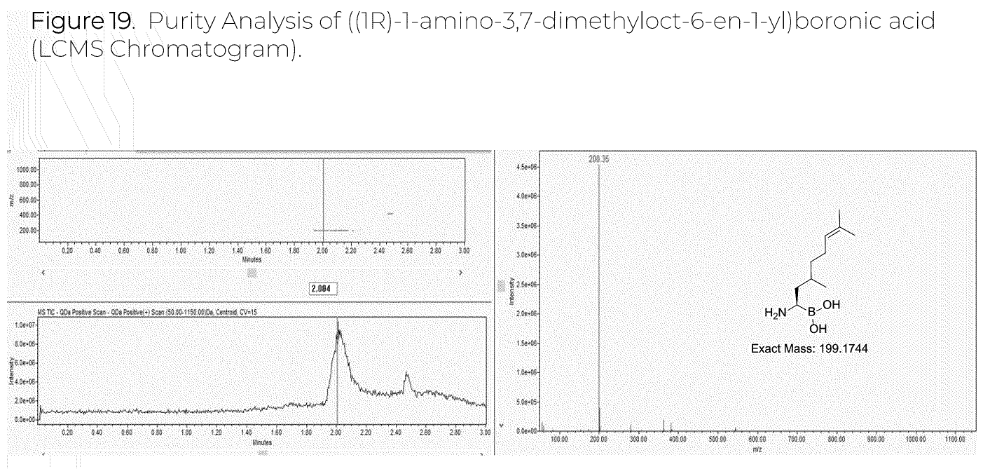

((1R)-1-amino-3,7-dimethyloct-6-en-1-yl)boronic acid was analyzed by LC/MS to confirm the molecular weight and the purity was determined after the preparation of aqueous solution. Briefly, (1R)-1-amino-3,7-dimethyloct-6-en-1-yl)boronic acid was analyzed using Luna Omega Polar C18 column (2.1×150 mm, Phenomenex) maintained at 40° C. and with the flow rate of 0.5 ml/min. The bound test article was eluted in the gradient of acetonitrile—0.1% TFA. The resulting purity analysis of ((1R)-1-amino-3,7-dimethyloct-6-en-1-yl)boronic acid is shown in FIG. 19:

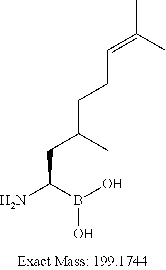

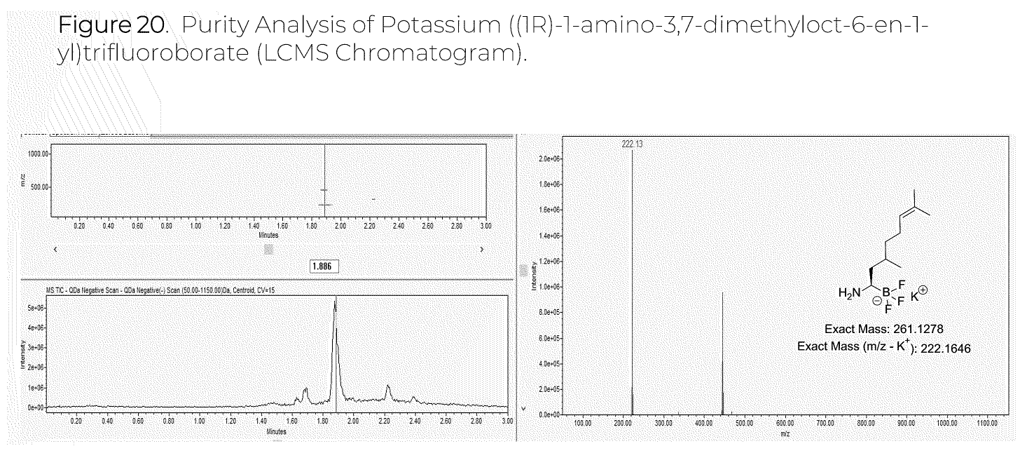

((1R)-1-amino-3,7-dimethyloct-6-en-1-yl)trifluoroborate was analyzed by LC/MS to confirm the molecular weight and the purity was determined after the preparation of aqueous solution. Briefly, ((1R)-1-amino-3,7-dimethyloct-6-en-1-yl)trifluoroborate was analyzed using Luna Omega Polar C18 column (2.1×150 mm, Phenomenex) maintained at 40° C. and with the flow rate of 0.5 ml/min. The bound test article was eluted in the gradient of acetonitrile—0.1% TFA. The resulting purity analysis of ((1R)-1-amino-3,7-dimethyloct-6-en-1-yl)trifluoroborate is shown in FIG. 20:

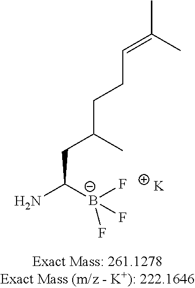

### Example 6: Evaluation and Comparison of BPA and BF₃ Compound(s) in FaDu Cells In Vitro

In this experiment, the uptake of BPA, L-LBH, FBF and LBF was carried out to determine which compounds shows greater uptake FaDu cells. The following protocols were used. Briefly, FaDu cells were harvested and washed with PBS twice. The count was adjusted to 2 million/ml in HBSS. Boron compounds were added to cells at the final concentration of 2.5 mM in HBSS and the cells were incubated at 37° C., in a humidified 5% CO2 atmosphere for two (2) hrs. with shaking. Following a two (2) hour incubation, cells were harvested and washed twice with ice cold PBS. Cells were then suspended in 1 ml ice cold PBS and a portion (50 μl) was lysed in RIPA buffer and protein content determined by BCA assay. The remaining portion (950 μl) was subjected to nitric acid lysis (66.7% acid at 80° C.). Boron measurements were carried out using ICP-OES. Boronophenylalanine (as fructose solution) was used as a control.

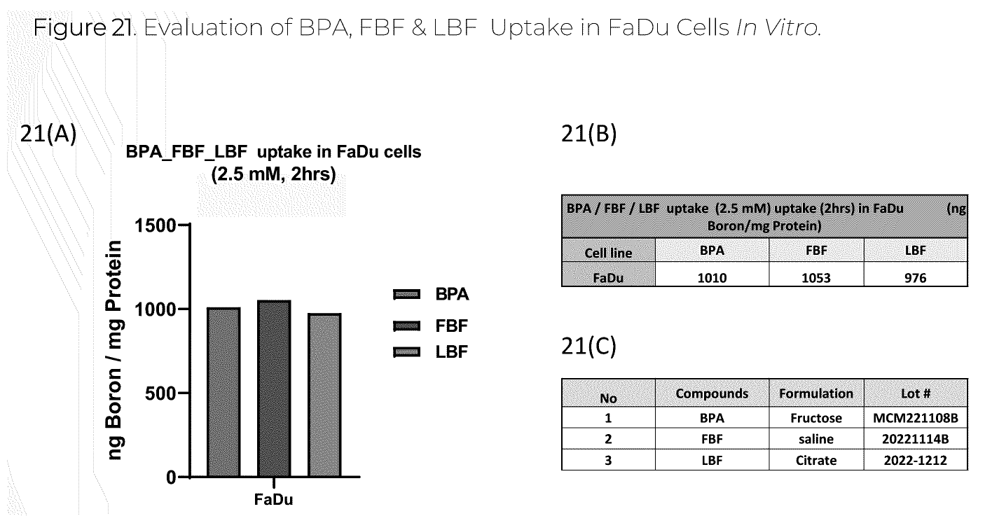

All compounds were taken up by FaDu cell, FBF and LBF being similar to BPA, and L-LBH lower. See, FIG. 21(A). Additionally, FIG. 21(B) shows numerically the amount of boron taken up in ng Boron/mg Protein at two (2) hours. FIG. 21(C) summarizes the compounds and the formulation used to make stock solutions.

### Example 7: Evaluation and Comparison of BPA and BF₃ Compound(s) in FaDu and U343MG Cells In Vitro

In this experiment, the uptake of BPA, FBH FBF, and LBH was carried out to determine which compounds show greater uptake FaDu and U343MG cells. The following protocols were used. Briefly, two (2) assays were performed, one using FaDu cells and the other using U343MG cells. FaDu and U343MG cells were harvested and washed with PBS twice. The count was adjusted to 2 million/ml in HBSS. Boron compounds were added to cells at the final concentration of 2.5 mM in HBSS and the cells were incubated at 37° C., in a humidified 5% CO2 atmosphere for two (2) hrs. with shaking. Following a two (2) hour incubation, cells were harvested and washed twice with ice cold PBS. Cells were then suspended in 1 ml ice cold PBS and a portion (50 μl) was lysed in RIPA buffer and protein content determined by BCA assay. The remaining portion (950 μl) was subjected to nitric acid lysis (66.7% acid at 80° C.). Boron measurements were carried out using ICP-OES. Boronophenylalanine (as fructose solution) was used as a control.

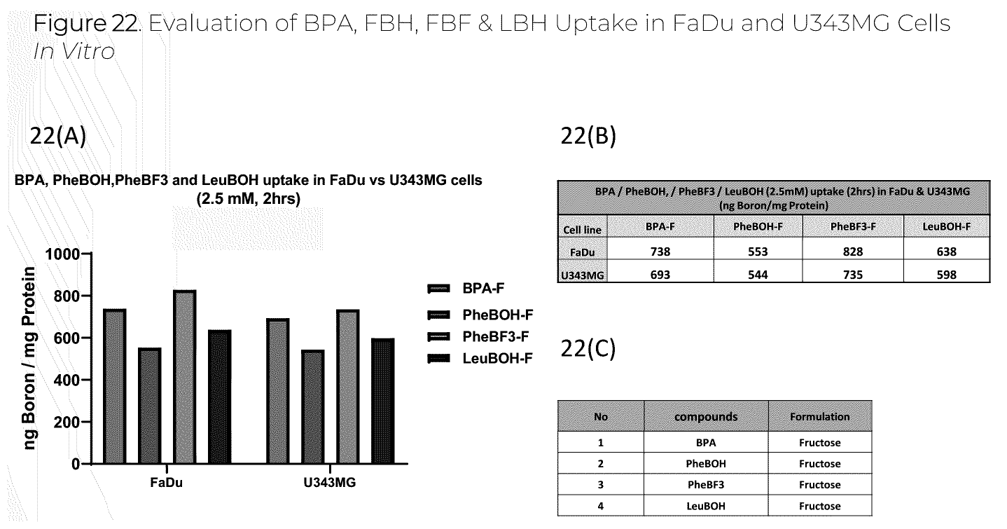

The results show that BPA, FBH FBF, and LBH have sufficient level uptake in U343MG cells but slightly lower uptake in FaDu cells. FBF has marginally greater uptake than the other compounds in both FaDu and U343MG cells. See, FIG. 22(A). Additionally, FIG. 22(B) shows numerically the amount of boron taken up in ng Boron/mg Protein at two (2) hours. FIG. 22(C) summarizes the compounds and the formulation used to make stock solutions.

### Example 8: Total Synthesis of Bortezomib (Using LBH as a Synthon)

Bortezomib was synthesized in the following manner using LBH as a Synthon. Briefly, to a solution of 30 mL of methylene chloride (DCM) is added 2 g of isovaleraldehyde and 9.7 mL of titanium ethoxide. After five (5) minutes of stirring, 2.81 g of (R)-(−)-tert-butylsulfinamide is added to the reaction. The reaction is refluxed at 40° C. for three (3) hours, then the temperature was brought down to 30° C. and allowed to stir overnight. After heating, the reaction is complete as observed by LCMS. Magnesium sulfate was added to the reaction and allowed to stir for five (5) minutes. The reaction mixture was then filtered through celite and concentrated to afford the target material:

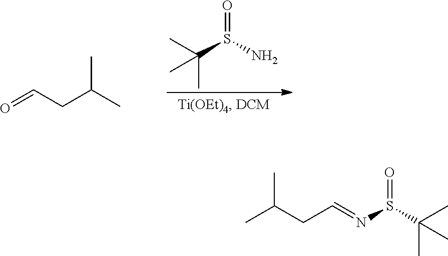

Then, to a solution of 5 mL of toluene at room temperature is added 70 mg of copper II sulfate pentahydrate (CuSO₄·5H₂O), 127 μL of benzylamine (BnNH₂), and 98 mg of tricyclohexylphosphonium tetrafluoroborate (Cy₃PHBF₄). After stirring for 15 minutes, an additional 40 mL of toluene was added followed by 4.4 g of (R,E)-2-methyl-N-(3-methylbutylidene)propane-2-sulfinamide and 11.8 g of bis(pinacolato)diboron. After stirring for twelve (12) hours, the reaction is complete as observed by LCMS. The reaction mixture is then diluted with 50 mL of ethyl acetate (EtOAc) and filtered through a silica plug. The solvent is removed under reduced pressure and the concentrate purified via flash column chromatography (FCC) to yield the target material:

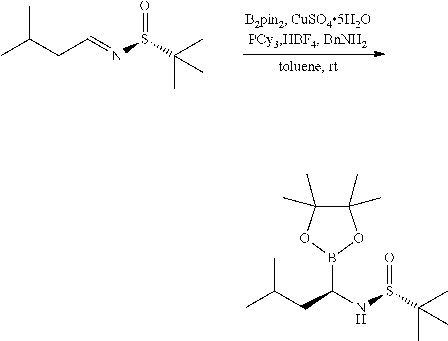

Then, to a solution containing 60 mL of methanol (MeOH) at −83° C. is added 6 g of (R)-2-methyl-N-[(1R)-3-methyl-1-(4,4,5,5-tetramethyl-1,3,2-dioxaborolan-2-yl)butyl]propane-2-sulfinamide. Next, 9.5 mL of 4M hydrochloric acid (HCl) in 1,4-dioxane was slowly added to the reaction. After two (2) hours of stirring, the reaction is complete as observed by LCMS. The methanol was then removed under reduced pressure and the concentrate was diluted with saturated sodium bicarbonate and ethyl acetate. The aqueous layer was then separated and placed onto a preparative HPLC to afford the following target material:

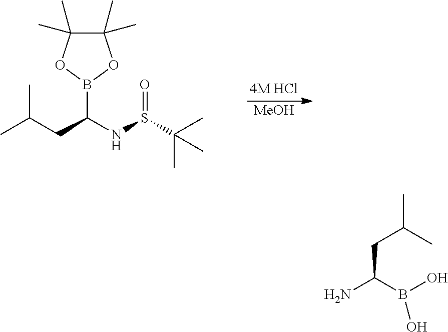

Then, to a solution containing 10 mL of methylene chloride (DCM) is added 1 g of pyrazinoic acid, 1.68 mL of N,N-diisopropylethylamine (DIPEA), and 3.1 g of [(1,2,3-benzotriazol-1-yloxy)(dimethylamino)methylidene]dimethylazanium tetrafluoroborate (TBTU). After ten (10) minutes of stirring, 1.5 g of L-phenylalanine is added. After twelve (12) hours of stirring, the reaction is complete as observed by LCMS. The methylene chloride was then removed under reduced pressure and placed onto a preparative HPLC to afford the following target material:

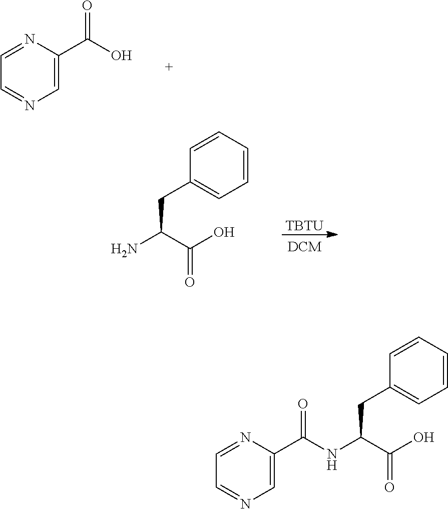

Finally, to a flask containing 227 mg of 5-{[(1S)-1-carboxy-2-phenylethyl]carbamoyl}pyrazine-2-ylium is added 7.6 mL of methylene chloride (DCM), 117 μL of trimethylamine (TEA), and 294 mg of [(1,2,3-benzotriazol-1-yloxy)(dimethylamino)methylidene]dimethylazanium tetrafluoroborate (TBTU). After fifteen (15) minutes of stirring, 100 mg of L-leucine boronic acid (LBH) is added. After twelve (12) hours of stirring, the reaction is complete as observed by LCMS. The methylene chloride (DCM) is then removed under reduced pressure and placed onto a preparative HPLC to afford the following target material Bortezomib in the annulated form:

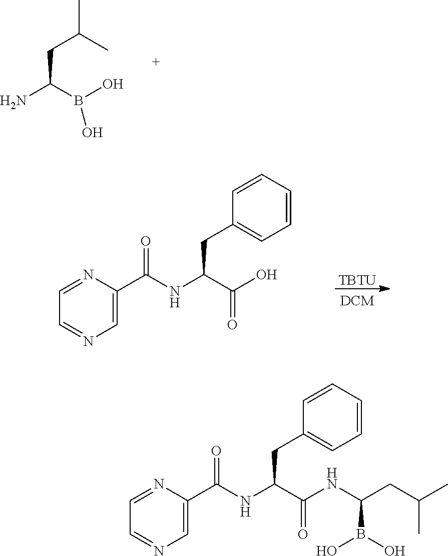

The resulting synthesis and composition denoted Bortezomib is shown in FIG. 24 and is set forth below:

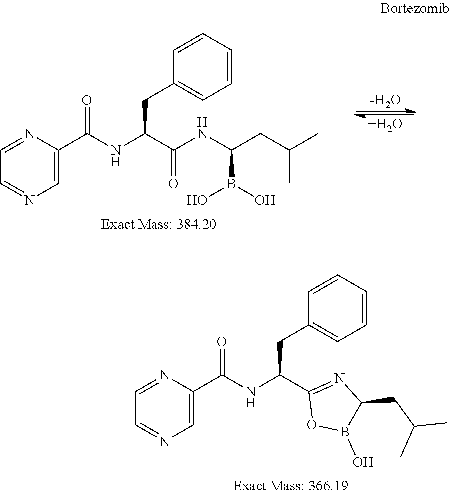

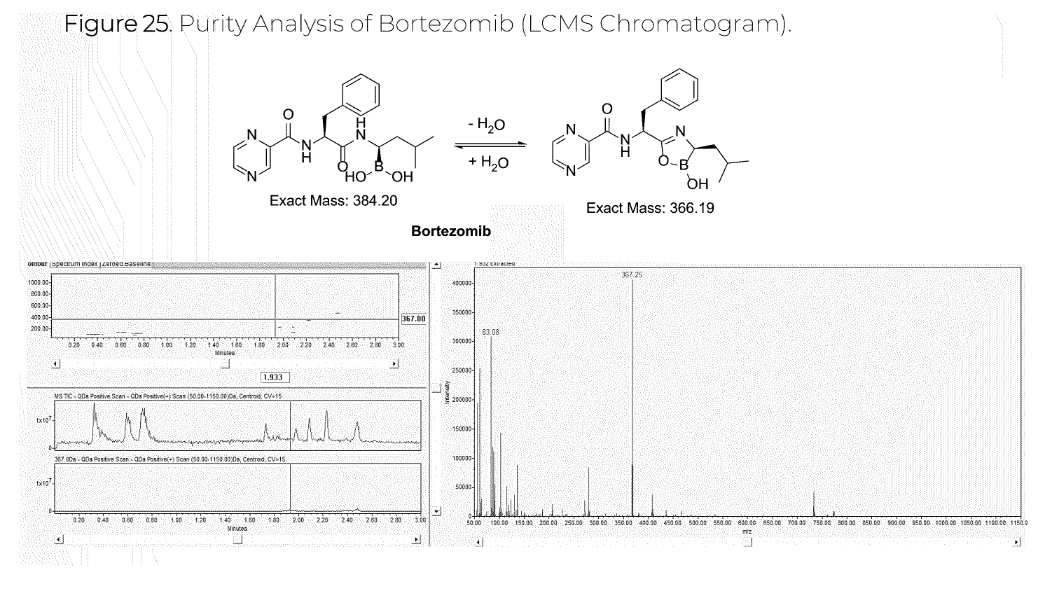

Bortezomib was analyzed by LC/MS to confirm the molecular weight and the purity was determined after the preparation of aqueous solution. Briefly, Bortezomib was analyzed using Luna Omega Polar C18 column (2.1×150 mm, Phenomenex) maintained at 40° C. and with the flow rate of 0.5 ml/min. The bound test article was eluted in the gradient of acetonitrile—0.1% TFA. The resulting purity analysis of Bortezomib is shown in FIG. 25.

### Example 9: Human Clinical Trials for the Treatment of Human Carcinomas Through the Use of BF₃ Compounds

BF₃ compounds are synthesized in accordance with the present invention which specifically accumulate in a tumor cell and are used in the treatment of certain tumors and other immunological disorders and/or other diseases. In connection with each of these indications, two clinical approaches are successfully pursued.

I.) Adjunctive therapy: In adjunctive therapy, patients are treated with BF₃ compounds in combination with a chemotherapeutic or pharmaceutical or biopharmaceutical agent or a combination thereof. Primary cancer targets are treated under standard protocols by the addition of BF₃ compounds and then irradiated or are treated with BF₃ compounds as a single agent. Protocol designs address effectiveness as assessed by the following examples, including but not limited to, reduction in tumor mass of primary or metastatic lesions, increased progression free survival, overall survival, improvement of patient's health, disease stabilization, as well as the ability to reduce usual doses of standard chemotherapy and other biologic agents. These dosage reductions allow additional and/or prolonged therapy by reducing dose-related toxicity of the chemotherapeutic or biologic agent.

II.) Monotherapy: In connection with the use of the BF₃ compounds in monotherapy of tumors, the BF₃ compounds are administered to patients without a chemotherapeutic or pharmaceutical or biological agent. In one embodiment, monotherapy is conducted clinically in end-stage cancer patients with extensive metastatic disease. Protocol designs address effectiveness as assessed by the following examples, including but not limited to, reduction in tumor mass of primary or metastatic lesions, increased progression free survival, overall survival, improvement of patient's health, disease stabilization, as well as the ability to reduce usual doses of standard chemotherapy and other biologic agents.

### Dosage

Dosage regimens may be adjusted to provide the optimum desired response. For example, a single BF₃ compound injection may be administered, several divided doses may be administered over time, or the dose may be proportionally reduced or increased as indicated by the exigencies of the therapeutic situation. “Dosage Unit Form” as used herein refers to physically discrete units suited as unitary dosages for the mammalian subjects to be treated; each unit containing a predetermined quantity of active compound calculated to produce the desired therapeutic effect in association with the required pharmaceutical carrier. The specification for the dosage unit forms of the invention is dictated by and directly dependent on (a) the unique characteristics of the BF₃ compounds, the individual mechanics of the irradiation mechanism (reactor) and the particular therapeutic or prophylactic effect to be achieved, and (b) the limitations inherent in the art of compounding such a compound for the treatment of sensitivity in individuals.

### Clinical Development Plan (CDP)

The CDP follows and develops treatments of cancer(s) and/or immunological disorders using BF₃ compounds of the disclosure which are then irradiated using Neutron Capture Therapy in connection with adjunctive therapy or monotherapy. Trials initially demonstrate safety and thereafter confirm efficacy in repeat doses. Trials are open label comparing standard chemotherapy with standard therapy plus BF₃ compounds which are then irradiated using Boron Neutron Capture Therapy. As will be appreciated, one non-limiting criteria that can be utilized in connection with enrollment of patients is concentration of BF₃ compounds in a tumor as determined by standard detection methods known in the art.

The present invention is not to be limited in scope by the embodiments disclosed herein, which are intended as single illustrations of individual aspects of the invention, and any that are functionally equivalent are within the scope of the invention. Various modifications to the models, methods, and life cycle methodology of the invention, in addition to those described herein, will become apparent to those skilled in the art from the foregoing description and teachings, and are similarly intended to fall within the scope of the invention. Such modifications or other embodiments can be practiced without departing from the true scope and spirit of the invention.

TABLE I
Naturally Occuring Amino Acids.

| SINGLE LETTER | THREE LETTER | FULL NAME |
|---|---|---|
| F | Phe | phenylalanine |
| L | Leu | leucine |
| S | Ser | serine |
| Y | Tyr | tyrosine |
| C | Cys | cysteine |
| W | Trp | tryptophan |
| P | Pro | proline |
| H | His | histidine |
| Q | Gln | glutamine |
| R | Arg | arginine |
| I | Ile | isoleucine |
| M | Met | methionine |
| T | Thr | threonine |
| N | Asn | asparagine |
| K | Lys | lysine |
| V | Val | valine |
| A | Ala | alanine |
| D | Asp | aspartic acid |
| E | Glu | glutamic acid |
| G | Gly | glycine |

1) A method of producing a composition having the following chemical structure: 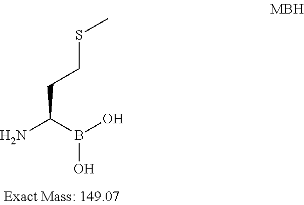
wherein the composition is produced by the process set forth in Example 3.

2) A method of producing a composition having the following chemical structure: 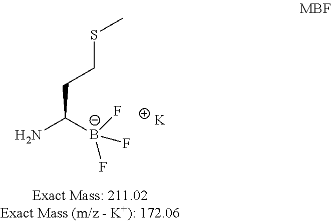
wherein the composition is produced by the process set forth in Example 3.

3) A method of producing a composition having the following chemical structure: 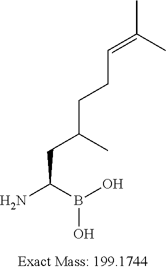
wherein the composition is produced by the process set forth in Example 5.

4) A method of producing a composition having the following chemical structure: 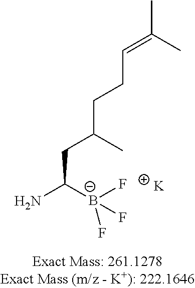
wherein the composition is produced by the process set forth in Example 5.

5) A kit comprising the composition of claim 1.

6) A kit comprising the composition of claim 2.

7) A kit comprising the composition of claim 3.

8) A kit comprising the composition of claim 4.

9) A composition produced by the method of claim 1.

10) A composition produced by the method of claim 2.

11) A composition produced by the method of claim 3.

12) A composition produced by the method of claim 4.

13) A Dosage Unit form comprising a composition of claim 6.

14) A Dosage Unit form comprising a composition of claim 7.

15) A Dosage Unit form comprising a composition of claim 8.

16) A Dosage Unit form comprising a composition of claim 9.

17) A method of performing Neutron Capture Therapy in the treatment of human cancer comprising:
a. synthesizing a Human Unit Dose of a BF₃ Compound;
b. injecting the BF₃ Compound into a tumor, whereby the BF₃ Compound accumulates into a cell; and
c. irradiating the BF₃ Compound with neutrons.

18) The method of claim 17, wherein the composition is selected from the group consisting of the compositions in claim(s) 6, 7, 8, and 9.

19) The method of claim 17, wherein the Neutron Capture Therapy is Boron Neutron Capture Therapy.

20) The method of claim 17, wherein the irradiation comprises epithermal neutrons.
